(19) United States
(12) Patent Application Publication
(10) Pub. No.: US 2020/0038052 A1
(43) Pub. Date: Feb. 6, 2020

(54) ULTRASONIC SURGICAL INSTRUMENT, ULTRASONIC SURGICAL ASSEMBLY AND A METHOD THEREOF

(71) Applicant: Olympus Corporation, Tokyo (JP)

(72) Inventors: Ken Fujisaki, Sagamihara-shi (JP); Takamitsu Sakamoto, Tokyo (JP); Ken Yokoyama, Tokyo (JP); Michio Takayama, Tokyo (JP)

(73) Assignee: Olympus Corporation, Tokyo (JP)

(21) Appl. No.: 16/582,724

(22) Filed: Sep. 25, 2019

Related U.S. Application Data
(63) Continuation of application No. PCT/JP2018/013928, filed on Mar. 30, 2018.
(60) Provisional application No. 62/479,567, filed on Mar. 31, 2017.

(51) Int. Cl. A61B 17/32; A61B 17/16

(52) U.S. Cl. CPC A61B 17/320068; A61B 2017/32007; A61B 2017/320069; A61B 17/1637

(57) ABSTRACT

An ultrasonic surgical instrument includes an elongated shaft having respective proximal and distal end sides. An ultrasonic transducer is secured to the shaft and is configured to transmit ultrasonic vibrations from the proximal end side toward a distal end side along a longitudinal axis of the shaft. A cutting portion includes an outermost shape-defining portion that when pressed at a tip of the block shape against a bone cuts the bone in a direction of a pressing force to form a bone tunnel and to produce small particle of bone. A burying portion is configured to direct a flow of the fluid, which contains the small particle of bone produced upon formation of the bone tunnel, toward a wall formed in the bone tunnel by the cutting portion along the direction of the pressing force and to bury the small particle of bone in the wall of the bone tunnel.

### CROSS-REFERENCE TO RELATED APPLICATIONS

This application is a continuation application of PCT Application No. PCT/JP2018/013928 filed on Mar. 30, 2018, which in turn claim priority to the U.S. Provisional Application No. 62/479567 filed on Mar. 31, 2017 which is hereby incorporated by reference in its entirety.

### TECHNICAL FIELD

The disclosed technology relates generally to an ultrasonic surgical system, and more particularly, some embodiments relate to an ultrasonic surgical instrument and ultrasonic surgical assembly, which perform mechanical ablation by ultrasonic vibrations.

### DESCRIPTION OF THE RELATED ART

In general, surgery involving treatment for the formation of bone tunnels in bone, for example, knee joint surgery includes anterior cruciate ligament (ACL) reconstruction surgery that replaces a damaged ligament with an alternative ligament or tendon graft. In this anterior cruciate ligament reconstruction surgery, treatment is applied to bones, to which an alternative ligament is to be grafted, to form bone tunnels. According to the current surgical procedure, an instrument having a rotary blade, for example, a drill is used, and the rotating drill is advanced to form a bone tunnel that is circular in cross-section.

Early healing of a tendon graft in bone tunnels is desired. For example, Japanese Patent Laid-Open No. 2003-320014 discloses that healing is promoted by applying an impregnation treatment of small particle of bone, which has been formed upon formation of a bone tunnel, to a wall surface of the bone tunnel. Japanese Patent Laid-Open No. 2003-320014 also discloses that the small particle of bone desirably has a diameter or particle size enabling its rapid absorption by macrophages, specifically 50 μm or smaller, with 20 μm or smaller being preferred.

If a drill is used for the formation of a bone tunnel, the resulting small particle of bone is discharged of the bone tunnel. A need therefore arises to collect the resulting small particle of bone if the treatment is assumed to be applied to push in the wall surface of the bone tunnel with the small particle of bone. Otherwise, the application of the treatment described hereinbefore needs to conduct treatment steps of taking a bone, which has been removed from another site different from the bone tunnel, out of the body, crushing the bone to produce small particle of bone, and then applying the small particle of bone to the wall surface of the bone tunnel or a treatment site.

### BRIEF SUMMARY OF EMBODIMENTS

The disclosed technology has been made in view of the foregoing. The disclosed technology provides an ultrasonic surgical instrument and ultrasonic surgical assembly, which transmits ultrasonic vibrations and can form a bone tunnel while producing small particle of bones in the bone tunnel.

Accordingly, one aspect of the disclosed technology is directed to an ultrasonic surgical instrument comprising an elongated shaft having respective proximal and distal end sides. An ultrasonic transducer is secured to the shaft via the proximal end side. The ultrasonic transducer configured to generate and to transmit ultrasonic vibrations from the proximal end side toward a distal end side along a longitudinal axis of the shaft. A cutting portion is disposed on the distal end side of the shaft. The cutting portion includes an outermost shape-defining portion that defines an outermost shape having a block shape that when pressed at a tip of the block shape against a bone while in a fluid with the ultrasonic vibrations being transmitted thereto, cuts the bone in a direction of a pressing force to form a bone tunnel and to produce small particle of bone at the tip. A burying portion is disposed on the proximal end side of the outermost shape-defining portion and is configured to direct a flow of the fluid, which contains the small particle of bone produced upon formation of the bone tunnel, toward a wall formed in the bone tunnel by the cutting portion along the direction of the pressing force and to bury the small particle of bone in the wall of the bone tunnel.

Another aspect of the disclosed technology is directed to an ultrasonic surgical assembly comprising an ultrasonic surgical instrument having an elongated shaft including respective proximal and distal end sides. An ultrasonic transducer is secured to the shaft via the proximal end side. The ultrasonic transducer is configured to generate and to transmit ultrasonic vibrations from the proximal end side toward a distal end side along a longitudinal axis of the shaft. A cutting portion is disposed on the distal end side of the shaft. The cutting portion includes an outermost shape-defining portion that defines an outermost shape having a block shape that when pressed at a tip of the block shape against a bone while in a fluid with the ultrasonic vibrations being transmitted thereto, cuts the bone in a direction of a pressing force to form a bone tunnel and to produce small particle of bone at the tip. A burying portion is disposed on the proximal end side of the outermost shape-defining portion and is configured to direct a flow of the fluid, which contains the small particle of bone produced upon formation of the bone tunnel toward a wall formed in the bone tunnel by the cutting portion along the direction of the pressing force and to bury the small particle of bone in the wall of the bone tunnel. An ultrasonic vibration generating section is attached on a proximal end side of the ultrasonic surgical instrument.

A further aspect of the disclosed technology is directed to a method of operating an ultrasonic surgical instrument for forming a bone tunnel using an ultrasonic device having a shaft. The shaft includes a distal end and a proximal end and a treatment portion disposed on the distal end of the shaft. The method is comprising: holding the treatment portion in contact with a bone to determine a position where the bone tunnel is to be formed; causing the treatment portion to vibrate in a direction of a longitudinal axis of the shaft while holding the treatment portion in contact with the bone, thereby the bone tunnel is formed; and producing burying small particle of bone upon formation of the bone tunnel in a bone wall at an area of a surface forming the bone tunnel wherein the area extending along a direction of vibrations of the treatment portion.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a schematic view depicting an ultrasonic surgical system to which first to tenth embodiments can be applied.

FIG. 2A is a schematic view illustrating a state in which a bone tunnel is being formed in a bone by a cutting portion of an ultrasonic surgical instrument according to the first embodiment.

FIG. 2B is a schematic view depicting an external appearance of the cutting portion of the ultrasonic surgical instrument when the cutting portion is seen from a direction of arrow 2B in FIG. 2A.

FIG. 2C is an enlarged schematic view of a position indicated by numeral reference 2C in FIG. 2A.

FIG. 2D is a schematic view depicting a modification of the external appearance of the cutting portion of the ultrasonic surgical instrument when the cutting portion is seen from the direction of arrow 2B in FIG. 2A.

FIG. 3A is a schematic view illustrating a state in which a bone tunnel is being formed in a bone by a cutting portion of an ultrasonic surgical instrument according to the second embodiment.

FIG. 3B is an enlarged schematic view of a position indicated by numeral reference 3B in FIG. 3A.

FIG. 4A is a schematic view illustrating a state in which a bone tunnel is being formed in a bone by a cutting portion of an ultrasonic surgical instrument according to the third embodiment.

FIG. 4B is an enlarged schematic view of a position indicated by numeral reference 4B in FIG. 4A.

FIG. 5A is a schematic view illustrating a state in which a bone tunnel is being formed in a bone by a cutting portion of an ultrasonic surgical instrument according to the fourth embodiment.

FIG. 5B is an enlarged schematic view of a position indicated by numeral reference 5B in FIG. 5A.

FIG. 6A is a schematic view depicting a cutting portion and a tip portion of a probe main body of an ultrasonic surgical instrument according to the fifth embodiment.

FIG. 6B is a schematic view illustrating how a cutting fluid and small particle of bone are moving when a bone tunnel is being formed in a bone by the cutting portion of the ultrasonic surgical instrument according to the fifth embodiment. The cutting fluid comprising a cerebrospinal fluid in bone, the small particle of bone, and a perfusate.

FIG. 7 is a schematic view depicting a cutting portion and a tip portion of a probe main body of an ultrasonic surgical instrument according to the sixth embodiment.

FIG. 8A is a schematic view illustrating a state in which a bone tunnel is being formed in a bone by a cutting portion of an ultrasonic surgical instrument according to the seventh embodiment.

FIG. 8B is an enlarged schematic view of a position indicated by numeral reference 8B in FIG. 8A.

FIG. 8C is an enlarged schematic view illustrating a state in which by cavitation caused in a recessed portion of the cutting portion depicted in FIG. 8A, a flow of the cutting fluid and small particle of bone, the flow going from the recessed portion toward a side wall of a bone tunnel, is induced and the small particle of bone is being buried in the side wall of the bone tunnel.

FIG. 9 is a schematic view depicting a modification of FIG. 8B in a state in which a porous Ti filter is disposed on a flange at the position indicated by numeral reference 8B in FIG. 8A.

FIG. 10 is a schematic view illustrating a state in which a bone tunnel is being formed in a bone by a cutting portion of an ultrasonic surgical instrument according to the eighth embodiment.

FIG. 11 is a schematic view illustrating a state in which a bone tunnel is being formed in a bone by a cutting portion of an ultrasonic surgical instrument according to the ninth embodiment.

FIG. 12 is a schematic view illustrating a state in which a bone tunnel is being formed in a bone by a cutting portion of an ultrasonic surgical instrument according to the tenth embodiment.

FIG. 13 is a photo depicting a buried state of small particle of bone in a periphery of a bone tunnel after cutting by an ultrasonic probe.

FIG. 14 is a diagram or photo depicting a buried state of small particle of bone in a periphery of a bone tunnel after cutting by a drill.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

With reference to the drawings, a description will hereinafter be made about ultrasonic surgical instruments.

### First Embodiment

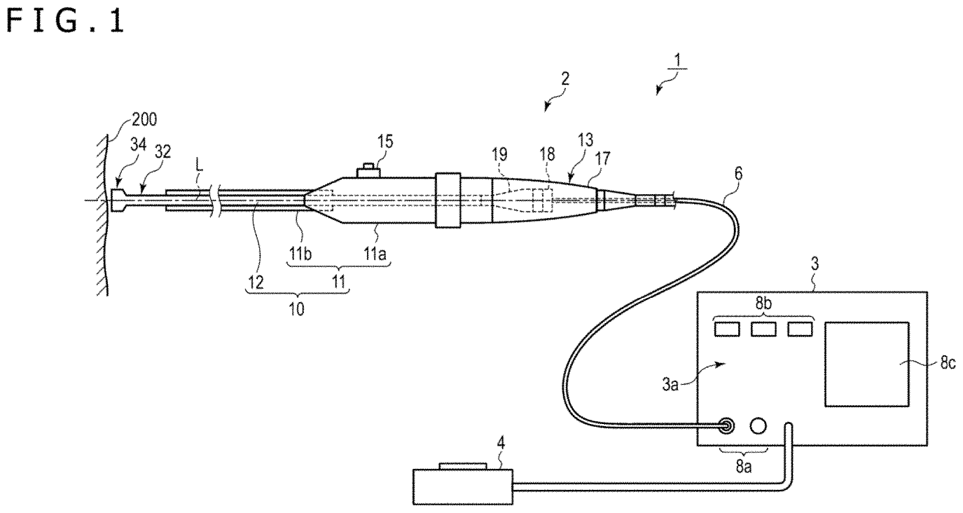

FIG. 1 is a view depicting an ultrasonic surgical system 1 according to a first embodiment.

The ultrasonic surgical system 1 of this embodiment primarily includes an ultrasonic surgical assembly 2, a power supply section 3, and a foot switch 4 that controls on/off of generation of ultrasonic vibrations. The ultrasonic surgical assembly 2 and the power supply section 3 are connected via a cable 6. Supply of drive power or energy and transmission and reception of control signals are hence performed between the ultrasonic surgical assembly 2 and the power supply section 3. On a front panel 3a of the power supply section 3, a plurality of connectors 8a for detachable connection to the ultrasonic surgical assembly 2 via the cable 6, various control switches 8b and a display screen 8c, which displays information needed for treatment, are disposed. The ultrasonic surgical system 1 is used in combination with an endoscope system and a perfusion apparatus to be provided additionally depending on surgical procedures and details. The endoscope system is used to take an image in a joint as needed by using an undepicted endoscope and to display the image on an undepicted monitor. The perfusion apparatus introduces a perfusate such as physiological saline into a joint capsule or the like via an appropriate portal to fill up the joint capsule with the perfusate, and discharges any unnecessary portion of the perfusate from the joint capsule or the like.

The ultrasonic surgical assembly 2 includes an ultrasonic surgical instrument 10, and an ultrasonic transducer unit or ultrasonic vibration generating section 13 fixed on a proximal end side of the ultrasonic surgical instrument 10. The ultrasonic surgical instrument 10 includes a device main body or exterior 11, and an ultrasonic probe or blade 12.

The ultrasonic surgical instrument 10 is formed in a cylindrical shape. The device main body 11 includes a housing 11a disposed extending through the ultrasonic probe 12, and a sheath 11b extending from the housing 11a to a distal end side along a longitudinal axis L and covering the ultrasonic probe 12 at a desired position thereof. The longitudinal axis L coincides with central axes of the device main body 11, ultrasonic probe 12 and ultrasonic transducer unit 13. Ultrasonic vibrations, which will be described hereinafter, are longitudinal vibrations to be transmitted from the proximal end side toward the distal end side along the longitudinal axis L.

The sheath 11b and ultrasonic probe 12 may be detachably attached to the housing 11a.

On the housing 11a, a control switch 15 is disposed to control on/off of ultrasonic vibrations by a finger operation. The control switch 15 includes a function equivalent to the foot switch. Either the control switch 15 or the foot switch 4 may be used accordingly.

The ultrasonic transducer unit 13 is detachably attached to the housing 11a. The ultrasonic transducer unit 13 includes a transducer casing 17, an ultrasonic transducer 18 accommodated inside the casing 17, and a horn 19 accommodated inside the casing 17. The ultrasonic transducer 18 is formed of a piezoelectric device or the like, and generates ultrasonic vibrations when supplied with electric power or energy. The transducer 18 generates longitudinal ultrasonic vibrations of a predetermined resonant frequency along the longitudinal axis L. The transducer 18 is connected to a proximal end side of the horn 19 along the longitudinal axis L. The ultrasonic probe 12 is connected to a distal end side of the horn 19 along the longitudinal axis L. The horn 19 transmits ultrasonic vibrations, which have been generated at the ultrasonic transducer 18, along the longitudinal axis L to the ultrasonic probe 12 on its proximal end side while enlarging the amplitude of the ultrasonic vibrations. With the ultrasonic transducer unit 13 fitted to the housing 11a, a proximal end side of the ultrasonic probe 12 and the distal end side of the horn 19 are sonically connected together. Vertical ultrasonic vibrations generated at the ultrasonic transducer 18 are therefore transmitted from a proximal end of the ultrasonic probe 12 to a cutting portion 34 on a distal end of the ultrasonic probe 12.

The surgical assembly 2 illustrated in FIG. 1 includes no perfusate supply and discharge system. Although not illustrated in the figure, a perfusate supply and discharge system may be arranged in the surgical assembly 2 itself.

The ultrasonic probe 12 includes a probe main body or shaft 32 formed in a shaft shape, and the cutting portion 34 formed in a block shape. The ultrasonic probe 12 is formed from a metal material having good acoustic characteristics and sufficient hardness relative to a bone 200, for example, a titanium alloy or the like.

The probe main body 32 is detachably fixed at a proximal end thereof to a distal end of the horn 19. To a proximal end side of the probe main body or shaft 32 of the ultrasonic probe 12, the ultrasonic transducer 18 is secured to generate ultrasonic vibrations. Ultrasonic vibrations generated at the ultrasonic transducer 18 are hence transmitted along the longitudinal axis L from the proximal end side toward s distal end side of the probe main body 32.

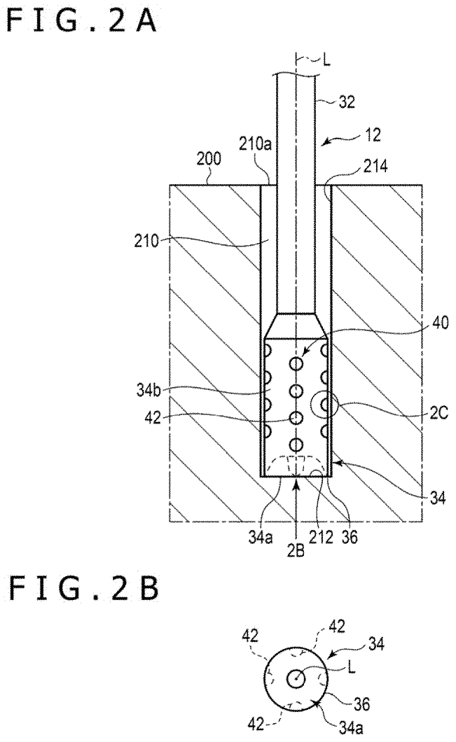

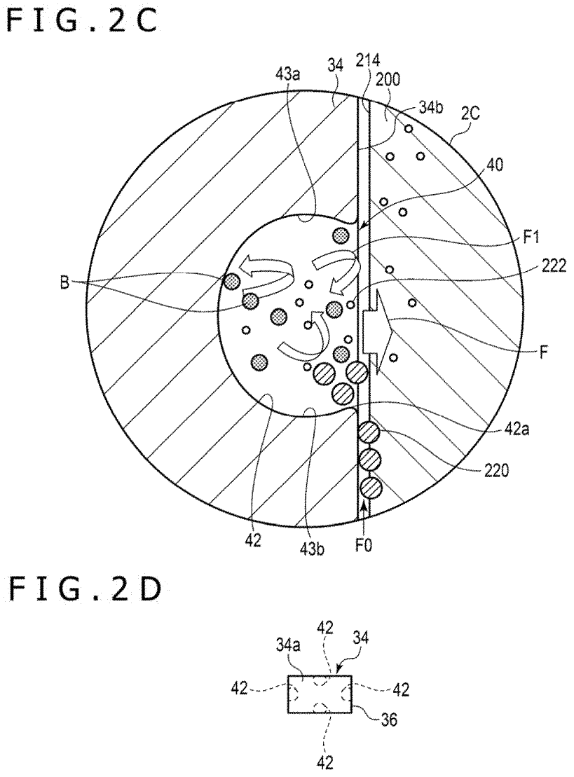

With ultrasonic vibrations being transmitted to the probe main body 32, the ultrasonic vibrations are also transmitted to the cutting portion 34. At this time, a tip or cutting surface 34a (see, for example, FIG. 2A) of the cutting portion 34, the tip or cutting surface 34a being to be described hereinafter, is pressed against the bone 200 in a fluid such as a perfusate to apply a hammering action the bone 200, so that the bone 200 is cut in a direction of a pressing force or in a moving direction of the cutting portion 34 to form a bone tunnel 210 (see, for example, FIG. 2A). The bone tunnel 210 includes an outline of opening 210a (see, for example, FIG. 2A), which has a similar shape and size as an outermost shape-defining portion 36 (see, for example, FIG. 2A) to be described hereinafter.

Suitably, the cutting of the bone 200 by the cutting portion 34 in a joint is conducted in its entirety in a fluid such as a cutting fluid that contains a perfusate and lipids to be described hereinafter. With ultrasonic vibrations being transmitted, the tip 34a of the cutting portion 34 induces a flow of the fluid such as the cutting fluid, which contains the perfusate and the like, in a direction along the vibrating direction of the ultrasonic vibrations.

The cutting portion 34 is disposed along the longitudinal axis L on the distal end side of the probe main body 32. Pressing of the cutting portion 34 at its tip 34a, which will be described hereinafter, against the bone 200 in the fluid with ultrasonic vibrations being transmitted to the probe main body 32 enables cutting of the bone 200 in the direction of the pressing force to form the bone tunnel 210. The cutting portion 34 is therefore used as an ablation end-effector to cut the bone 200 with ultrasonic vibrations. The bone 200 is primarily formed of a cortical bone on an outer side and a cancellous bone on an inner side. The bone tunnel 210 is formed to a depth where the bone tunnel 210 reaches the cancellous bone through the cortical bone. Specifically, the cancellous bone is formed in a spongy network structure.

FIG. 2A is a view illustrating the external shape of the cutting portion 34 of the ultrasonic surgical instrument 10 according to the first embodiment, and the bone tunnel 210 which is being formed by the cutting portion 34. FIG. 2B is a view depicting an external appearance of the tip 34a of the cutting portion 34 of the ultrasonic surgical instrument 10 when the cutting portion 34 is seen from a direction of arrow 2B in FIG. 2A. FIG. 2C is an enlarged schematic view of one of the recessed portions 42 disposed in the cutting portion 34, the one recessed portion 42 being indicated by numeral reference 2C in FIG. 2A. In the following description, in the axial direction or along the longitudinal axis L in or along which the ultrasonic probe 12 extends, a side closer to the housing 11a as illustrated in FIG. 1 is defined as a proximal end side, and a side toward which the longitudinal axis L extends as illustrated in FIG. 1 is defined as a distal end side.

The cutting portion 34 includes the tip or cutting surface 34a, and an outer circumferential surface 34b. In this embodiment, the tip or cutting surface 34a of the cutting portion 34 includes one or more apex portions or protruding portions extending toward the distal end side along the longitudinal axis L. The tip 34a of the cutting portion 34 may be pointed, or may be obtuse. In the example depicted in FIG. 2B, an outermost edge is formed in a circular shape, the inside of the outermost edge is formed as a circular inner edge that is located along the longitudinal axis L on the proximal end side of the outermost edge, and an inner side of the inner edge is formed as a pointed or cuspidal portion that is located along the longitudinal axis L on the distal end side of the inner edge. The cuspidal portion is formed in a pointed shape on the longitudinal axis L.

An appropriate step or steps may be formed on the tip 34a of the cutting portion 34 instead of the example depicted in FIG. 2B.

The cutting portion 34 includes an outermost shape-defining portion 36 defining an outermost shape when the proximal end side including the tip or cutting surface 34a is seen from the distal end side along the longitudinal axis L. In this embodiment, the outermost shape-defining portion 36 is formed over an appropriate length in parallel to the longitudinal axis L on the proximal end side relative to a proximal end of the tip 34a of the cutting portion 34.

As depicted in FIG. 2B, FIG. 2A illustrates an example in which the outer shape of the outermost shape-defining portion 36 of the cutting portion 34 is circular. The outer shape of the cutting portion 34 is substantially cylindrical, so that the bone tunnel 210 formed by the cutting portion 34 also has a substantially cylindrical outer shape. The cutting portion 34 can form the bone tunnel 210 with an opening in a form corresponding to the outermost shape-defining portion 36. In particular, the outline of opening 210a of the bone tunnel 210 is formed with a shape corresponding to the outermost shape-defining portion 36.

The bone tunnel 210 includes a bottom surface 212 and a side wall or wall 214. The bottom surface 212 has been formed by the tip 34a of the cutting portion 34, and is in contact with or opposes the tip 34a. The side wall or wall 214 has been formed by the outermost shape-defining portion 36, and is in contact with or opposes the outer circumferential surface 34b.

The bottom surface 212 of the bone tunnel 210 copies the outer shape of the tip 34a of the cutting portion 34. In this embodiment, a recess-protrusion configuration which corresponds to the outer shape of the tip 34a of the cutting portion 34 is therefore formed on the bottom surface 212. Accordingly, the bottom surface 212 of the bone tunnel 210 is not planar in this embodiment.

The side wall 214 of the bone tunnel 210 is formed along the moving direction of the cutting portion 34 or the direction of the pressing force in the bone tunnel 210 upon formation of the bone tunnel 210. In this embodiment, the cutting portion 34 is moved along the longitudinal axis L. The side wall 214 copies the outer shape of the outermost shape-defining portion 36. The side wall 214 is therefore formed as a columnar recess surface of an outer shape corresponding to the outer shape of the outermost shape-defining portion 36. Accordingly, the side wall 214 is formed as a cylindrical curved surface in this embodiment.

The cutting portion 34 includes a burying portion or retention-assisting portion 40 that by transmitted ultrasonic vibrations, directs a flow of the fluid or the cutting fluid, which contains small particle of bone 220 and 222 of various particle sizes formed upon formation of the bone tunnel 210, toward the side wall 214 to bury or retain the small particle of bone 220 and 222 in the side wall 214. The fluid contains marrow lipids in addition to the perfusate. The burying portion 40 is used as a flow-direction adjusting portion that deflects the flow of the cutting fluid or fluid, which is going in parallel or substantially parallel to the longitudinal axis L along the outer circumferential surface 34b of the cutting portion 34, from the direction parallel or substantially parallel to the longitudinal axis L to a direction in which the flow goes away toward a distal side relative to the cutting portion 34. The burying portion 40 therefore deflects the flow of the fluid in a direction different from the vibrating direction of ultrasonic vibrations.

Some offset is allowed between the vibrating direction of ultrasonic vibrations and the direction of a pressing force or the moving direction of the cutting portion 34. In other words, the bone tunnel 210 can be formed even if the moving direction of the cutting portion 34 is offset from the direction along the longitudinal axis L.

The burying portion 40 is disposed in the outer circumferential surface 34b of the cutting portion 34 at a location on the proximal end side of the tip 34a of the cutting portion 34. The burying portion 40 is disposed in the outer circumferential surface 34b of the cutting portion 34 at a location on the proximal side of a distal end of the outermost shape-defining portion 36. Accordingly, the burying portion 40 or recessed portions 42, which will be described hereinafter, are disposed in the outermost shape-defining portion 36.

The burying portion 40 includes the recessed portions 42 that extend inward of the cutting portion 34 relative to the outermost shape-defining portion 36. In this embodiment, each recessed portion 42 extends in a dome shape toward the longitudinal axis or central axis L relative to the contour of the outer circumferential surface 34b of the cutting portion 34.

Each recessed portion 42 is formed at a location on the proximal end side of the most distal position of the outermost shape-defining portion 36. In this embodiment, the recessed portions 42 are arranged side by side in the outer circumferential surface 34b of the cutting portion 34 at locations parallel to the longitudinal axis L. The individual recessed portions 42 are apart from each other by a suitable distance along the longitudinal axis L. In this embodiment, the recessed portions 42 are arranged side by side in plural rows along the longitudinal axis L in the outer circumferential surface 34b of the cutting portion 34. In this embodiment, the individual recessed portions 42 are formed at locations that are offset by approximately 90° about the longitudinal axis L.

Each recessed portion 42 includes an outline of opening portion or outer edge 42a that opposes the side wall 214 of the bone tunnel 210. The outline of opening portion 42a of the recessed portion 42 is formed with an outer shape and size so that a portion of the small particle of bone 220 and 222 produced upon formation of the bone tunnel 210 can be received in the recessed portion 42.

As illustrated in FIG. 2A, the recessed portions 42 in this embodiment will be described based on an example in which the recessed portions 42 are formed with a substantially even size and depth. However, the size and depth of each recessed portion 42 may be set as desired.

Now, the cutting portion 34 includes at the tip or cutting surface 34a thereof surfaces that intersect the vibrating direction of ultrasonic vibrations. If the tip 34a hits the bone 200 and the cutting fluid in the vibrating direction of ultrasonic vibrations at the tip 34a of the cutting portion 34 with ultrasonic vibrations being transmitted thereto, the bone 200 is crushed at a location where the bone 200 is in contact with or is close to the tip 34a of the cutting portion 34, and at the same time cavitation is caused. In association with a collapse of bubbles B, the cavitation further crushes the small particle of bone 220 so that the small particle of bone 220 is further crushed into finer particles. The cavitation hence progressively crushes the small particle of bone 220 of the bone 200 into still finer, particulate small particle of bone 222. By collision of the small particle of bone 220 itself, the small particle of bone 220 is also progressively crushed into still finer, particulate small particle of bone 222. The numeral references of the small particle of bone 220 and 222 are used for the sake of convenience, and the individual small particle of bone particles are different in particle size and shape.

As described hereinbefore, the cutting portion 34, with ultrasonic vibrations being transmitted thereto, deepens the bone tunnel 210 while crushing the bone 200 into fine particles. The bone tunnel 210 can be formed with a shape and size corresponding to the outer shape of the outermost shape-defining portion 36 of the cutting portion 34. The depth of the bone tunnel 210 can be set as desired.

When cavitation is caused at the tip 34a of the cutting portion 34, a flow, specifically a flow in a direction indicted by numeral reference F0 is induced by the cavitation in the cutting fluid. A portion of the cutting fluid is caused to flow, together with the crushed small particle of bone 220, according to the flow in the direction indicated by numeral reference F0 through a gap between the outer circumferential surface 34b of the cutting portion 34 and the side wall 214 of the bone tunnel 210. The portion of the cutting fluid and a portion of the crushed small particle of bone 220 therefore flow toward a proximal end side of the cutting portion 34. At this time, the small particle of bone 220 between the outer circumferential surface 34b of the cutting portion 34 and the side wall 214 of the bone tunnel 210 is progressively crushed into a still finer particulate form by the cavitation.

A portion of the small particle of bone 220, which has been crushed into the particulate form, and a portion of the cutting fluid flow into the recessed portion 42 through the outline of opening portion 42a.

FIG. 2C depicts an enlarged scale of a position indicated by numeral reference 2C in FIG. 2A. FIG. 2C depicts a cross-sectional shape of the bone tunnel 210 formed by the cutting portion 34 and a cross-sectional shape of one of the recessed portions 42 in the cutting portion 34 when the outer circumferential surface 34b of the cutting portion 34 has come close to or into contact with the side wall 214 of the bone tunnel 210.

The recessed portion 42 includes intersecting surfaces or cavitation-causing surfaces 43a and 43b which intersect the vibrating direction of ultrasonic vibrations or the axis parallel to the longitudinal axis L. At least portions of these intersecting surfaces 43a and 43b suitably intersect at right angle the vibrating direction of ultrasonic vibrations. It is therefore suited that the intersecting surfaces 43a and 43b intersect, preferably intersect at right angle the vibrating direction of ultrasonic vibrations and are formed as surfaces having areas which cause cavitation in the recessed portion 42. With ultrasonic vibrations being transmitted to the cutting portion 34, cavitation is caused at the intersecting surfaces 43a and 43b like the tip 34a of the cutting portion 34. In the recessed portion 42, a number of bubbles B are induced by the cavitation and a number of bubbles B are also caused to collapse by the cavitation. Therefore, a portion of the small particle of bone 220, which has flowed into the recessed portion 42, is crushed under the action of cavitation, and is progressively changed into still finer small particle of bone 222.

At this time, turbulent flows indicated by numeral reference F1 have occurred in the recessed portion 42.

A fraction of the bubbles remains without collapsing in the recessed portion 42 owing to the occurrence of cavitation, so that the volume of the cutting fluid in the recessed portion 42 rapidly increases. In the recessed portion 42, the pressure becomes higher than the pressure between the outer circumferential surface 34b of the cutting portion 34 and the side wall or wall 214 of the bone tunnel 210. Therefore, a portion of the cutting fluid and a portion of the fine small particle of bone 222 in the recessed portion 42 are therefore forced out in a direction as indicated by numeral reference F toward the side wall 214 of the bone tunnel 210.

Accordingly, the burying portion 40 or recessed portions 42 each deflect a flow of a portion of the cutting fluid and a portion of the small particle of bone 220 and 222 between the outline of opening portion 42a of the recessed portion 42 and the side wall 214 of the bone tunnel 210, the side wall 214 opposing the outline of opening portion 42a, in a direction different from the vibrating direction of ultrasonic vibrations. Here, the recessed portion 42 deflects the flow of the portion of the cutting fluid and the portion of the small particle of bone 220 and 222 between the outline of opening portion 42a of the recessed portion 42 and the side wall 214 of the bone tunnel 210, the side wall 214 opposing the outline of opening portion 42a, in a direction that intersects at right angle in the vibrating direction of ultrasonic vibrations. The portion of the cutting fluid and the portion of the small particle of bone 220 and 222 between the outline of opening portion 42a of the recessed portion 42 and the side wall 214 of the bone tunnel 210 and a portion of the cutting fluid and a portion of the small particle of bone 220 and 222 in the recessed portion 42 are therefore caused to flow toward the side wall 214 of the bone tunnel 210.

The cancellous bone in which the bone tunnel 210 is formed includes a network structure. The being flowed of the small particle of bone 220 and 222, which are finer than the network structure of the side wall 214 of the bone tunnel 210, together with the cutting fluid toward the side wall 214 of the bone tunnel 210 therefore results in adhesion and retention of a portion of the small particle of bone 220 and 222 in the network structure of the side wall 214 of the bone tunnel 210. Accordingly, a portion of the small particle of bone 220 and 222 ejected together with the cutting fluid from the recessed portion 42 is buried in the network structure of the side wall 214 of the bone tunnel 210. The portion of the small particle of bone 220 and 222 is retained in a vicinity of the surface of the side wall 214 of the bone tunnel 210.

Depending on the fineness of the network in the side wall 214 of the bone tunnel 210, even the small particle of bone 220, which is greater in particle size than the small particle of bone 222, is buried in the network structure of the side wall 214 of the bone tunnel 210.

As described hereinbefore, the recessed portion 42 disposed in the cutting portion 34, with ultrasonic vibrations being transmitted thereto, directs the flow of the fluid, which contains the small particle of bone 220 and 222 produced upon formation of the bone tunnel 210, toward the side wall 214 of the bone tunnel 210 by the ultrasonic vibrations. Accordingly, the burying portion 40 directs the flow of the fluid, which contains the small particle of bone 220 and 222 produced upon formation of the bone tunnel 210, toward the side wall 214, which extends along the direction of the pressing force or the moving direction of the probe 12 in the bone tunnel 210, and buries the small particle of bone 220 and 222 in the side wall 214 of the bone tunnel 210.

Here, the bone tunnel 210 is formed to an appropriate depth, so that the cutting portion 34, with ultrasonic vibrations being transmitted thereto, is moved along the direction of the pressing force or the moving direction of the probe 12. The position of each recessed portion 42 relative to the side wall 214 of the bone tunnel 210 therefore develops an offset as the bone tunnel 210 becomes deeper. At this time, the cutting portions 34 continues to cut the bottom surface 212 of the bone tunnel 210, and the recessed portion 42 continues to bury the small particle of bone 220 and 222 into the side wall 214. In the side wall 214 along the direction of the pressing force or the moving direction of the probe 12 in the bone tunnel 210, a region in which the small particle of bone 220 and 222 are buried is therefore formed in a linear pattern along the direction of the pressing force or the moving direction of the probe 12. In the side wall 214, the small particle of bone 220 and 222 are thus buried more at a location opposite the recessed portion 42 than a location which is not opposite the recessed portion 42.

In this embodiment, the recessed portions 42 are arranged side by side in plural rows which are parallel to the longitudinal axis L. At certain locations on the side wall 214 of the bone tunnel 210, the small particle of bone 220 and 222 are hence buried and retained over a plurality of times under the action of the recessed portions 42 based on movements of the cutting portion 34 relative to the bone tunnel 210.

The use of the ultrasonic surgical instrument 10 or surgical assembly 2 according to this embodiment can readily form the small particle of bone 220 and 222 in the bone tunnel 210 along with the formation of the bone tunnel 210. Further, the use of the ultrasonic surgical instrument 10 according to this embodiment can easily bury the small particle of bone 220 and 222, which are produced upon formation of the bone tunnel 210, in the side wall 214 of the bone tunnel 210 by the burying portion 40.

In expectation of promoted regeneration, it is a common practice to fill a defective part of a bone with small particle of bone produced by crushing bone pieces or fragments harvested from another site. In ACL reconstruction surgery, promoted healing can hence also be expected between the bone tunnel 210 and a bone part of an alternative ligament or tendon graft to be placed in the bone tunnel 210 if small particle of bone harvested upon formation of the bone tunnel 210 is filled in the side wall 214 of the bone tunnel 210.

If the surgical instrument 10 according to this embodiment is used, the small particle of bone 220 and 222 are automatically buried in the side wall 214 of the bone tunnel 210 during cutting of the bone 200. Accordingly, the use of the surgical instrument 10 according to this embodiment obviates the harvest of the small particle of bone 220 and 222 upon formation of the bone tunnel 210. In addition, the small particle of bone 220 and 222 can be filled in the side wall 214 of the bone tunnel 210 without taking the harvested small particle of bone 220 and 222 out of the body. Hence, harvesting of a new bone and crushing of the harvested bone are no longer needed, and small particle of bone can be aseptically buried in a surface of a healing part of a bone. It is, accordingly, possible to achieve shortening of time required for ACL reconstruction surgery.

In this embodiment, the vibrating direction and the longitudinal axis L coincide or substantially coincide each other. Relative to the position where the side wall 214 of the bone tunnel 210 is located, the position of each recessed portion 42 disposed in the cutting portion 34 with ultrasonic vibrations being transmitted thereto shifts as the cutting portion 34 moves in the depth direction of the bone tunnel 210. The cutting of the side wall 214 with the small particle of bone 220 and 222 buried therein is once suppressed at this time, because the outer circumferential surface 34b of the cutting portion 34, to which the ultrasonic vibrations are being transmitted, is cylindrical. A region in which the small particle of bone 220 and 222 are linearly buried in parallel to the longitudinal axis L, is hence formed in the side wall 214 of the bone tunnel 210.

The outer circumferential surface 34b of the cutting portion 34 includes rows in each of which the recessed portions 42 are arranged side by side along the longitudinal axis L and rows in each of which no recessed portions 42 are formed. Into the side wall 214 of the bone tunnel 210, the small particle of bone 220 is therefore buried in a greater amount, specifically in a greater small particle of bone amount per unit volume at locations corresponding to the rows in each of which the recessed portions 42 are arranged side by side than locations corresponding to the rows in each of which no recessed portions 42 are formed.

According to the ultrasonic surgical instrument 10 of this embodiment, the state of filling of the side wall 214 of the bone tunnel 210 with the small particle of bone 220 and 222 can be controlled by designing the number and density of the recessed portions 42 as described hereinbefore. The outer circumferential surface 34b of the cutting portion 34 in this embodiment is preferably a columnar surface parallel to the longitudinal axis L rather than a truncated conical surface. When forming the bone tunnel 210, the bone tunnel 210 is progressively deepened, and at the same time the small particle of bone 220 and 222 are progressively buried. If the cutting portion 34 is truncated conical and has a smaller cross-sectional diameter at its distal end side than its proximal end side, in other words, has a shape flaring from the distal end side toward the proximal end side, the bone tunnel 210 has a cross-sectional diameter that becomes gradually greater toward a proximal end side of the bone tunnel 210. If the cross-sectional diameter of the bone tunnel 210 becomes gradually greater, there is a potential risk that the side wall 214 of the bone tunnel 210 may be cut together with the buried small particle of bone 220 and 222. If the cutting portion 34 is truncated conical and includes a greater cross-sectional diameter at its distal end side than its proximal end side, on the other hand, the distance between the burying portion 40, in other words, each recessed portion 42 and the side wall 214 is greater than that in the case of the cutting portion 34 being columnar.

The outer shape of the outermost shape-defining portion 36 of the cutting portion 34 when the proximal end side of the cutting portion 34 is seen from its distal end side is not limited to a circular shape. As depicted in FIG. 2D, for example, the outer shape of the outermost shape-defining portion 36 of the cutting portion 34 may be rectangular. If the outer shape of the outermost shape-defining portion 36 is rectangular, the side wall 214 of the bone tunnel 210 is formed as a plurality of side surfaces of a rectangular prism. In this case, plural recessed portions 42 are suitably arranged side by side in a row along the longitudinal axis L in each side surface of the outer circumferential surface 34b of the cutting portion 34.

As the outer shape of the outermost shape-defining portion 36 of the cutting portion 34 when the proximal end side of the cutting portion 34 is seen from its distal end side, a variety of shapes such as elliptical shapes, polygonal shapes and athletics track shapes is allowable in addition to those depicted in FIGS. 2B and 2D. The bone tunnel 210 can, therefore, be formed in a desired shape by using an ultrasonic surgical instrument 10 that includes a cutting portion 34 including an outermost shape-defining portion 36 of a corresponding outer shape. The use of the ultrasonic surgical instrument 10 according to this embodiment can, therefore, appropriately bury the small particle of bone 220 and 222 in the side wall 214 of the bone tunnel 210 of the desired shape.

### Second Embodiment

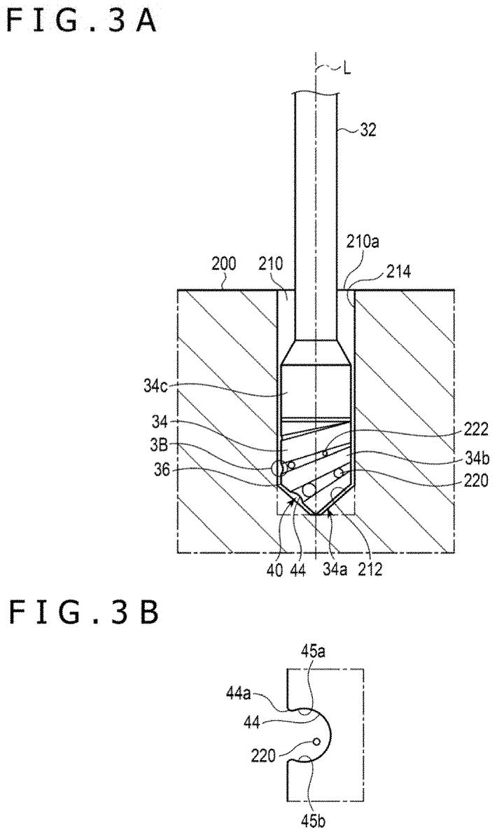

With reference to FIGS. 3A and 3B, a description will next be made about a second embodiment. This embodiment is a modification of the first embodiment. As the outer shape of a block-shaped cutting portion 34, desired shapes are allowable without being limited to a cylindrical shape. In an ultrasonic surgical instrument 10 of this embodiment, elements other than the cutting portion 34, such as the probe main body or shaft 32, for example, have similar configurations as in the first embodiment described hereinbefore, and therefore their description is omitted herein. In addition, detailed functions of the ultrasonic surgical instrument 10 are also similar to those described hereinbefore with respect to the first embodiment, and therefore their description is omitted herein.

FIG. 3A is a view illustrating the external shape of the cutting portion 34 of the ultrasonic surgical instrument 10 according to the second embodiment, and a bone tunnel 210 which is being formed by the cutting portion 34. FIG. 3B is an enlarged schematic view of one of recessed portions 44 disposed in the cutting portion 34 and indicated by numeral reference 3B in FIG. 3A.

As illustrated in FIG. 3A, the cutting portion 34 in the second embodiment is an example in which, when the cutting portion 34 is seen from its distal end side toward its proximal end side along a longitudinal axis L, an outermost shape-defining portion 36 is circular similar to the cutting portion 34 described with respect to the first embodiment. The outer shape of the cutting portion 34 and the outer shape of a bone tunnel 210 formed by the cutting portion 34 are substantially cylindrical. A burying portion 40 is disposed in the outer circumferential surface 34b of the cutting portion 34 at a location on the proximal end side of a tip 34a.

The tip 34a of the cutting portion 34 is formed substantially conical. Therefore, the bone tunnel 210 includes a bottom surface 212 that is formed conical.

The burying portion 40 is disposed in the outer circumferential surface 34b of the cutting portion 34 at the location on the proximal end side of the tip 34a. Accordingly, the burying portion 40 is disposed in the outer circumferential surface 34b of the cutting portion 34 at a location on the proximal end side of a tip of the outermost shape-defining portion 36. Accordingly, the burying portion 40 or recessed portions 44, which will be described hereinafter, are disposed in the outermost shape-defining portion 36.

The burying portion 40 disposed in the cutting portion 34 includes a recessed portion or recessed groove 44 that extends inward of the cutting portion 34 relative to the outermost shape-defining portion 36. In this embodiment, the recessed portion 44 extends toward the longitudinal axis or a central axis L relative to the contour of the outer circumferential surface 34b of the cutting portion 34. The recessed portion 44 is formed helical. In the outer circumferential surface 34b of the cutting portion 34, the recessed portion 44 is formed helical over a range of at least 360° about the longitudinal axis L.

The recessed portion 44 also continues to the tip 34a of the cutting portion 34, the tip 34a being on the distal end side of the outermost shape-defining portion 36. The recessed portion 44 is, therefore, continuously formed helical from the tip 34a or its vicinity of the cutting portion 34 toward the proximal end side of the cutting portion 34. The recessed portion 44 is used as a passage for the cutting fluid and small particle of bone 220 and 222.

The recessed portion 44 illustrated in FIG. 3A becomes gradually narrower in groove width and gradually shallower in depth from a starting end in a direction from the tip 34a toward the proximal end side of the cutting portion 34, and is closed at a terminating end.

As depicted in FIG. 3B, the recessed portion 44 is formed, for example, semi-circular or arcuate without any sharply bent surface at a bottom surface thereof.

The recessed portion 44 includes intersecting surfaces or cavitation-causing surfaces 45a and 45b which intersect the vibrating direction of ultrasonic vibrations or an axis parallel to the longitudinal axis L. At least portions of these intersecting surfaces 45a and 45b suitably intersect at right angle the vibrating direction of ultrasonic vibrations. It is therefore suited that the intersecting surfaces 45a and 45b intersect, preferably intersect at right angle the vibrating direction of ultrasonic vibrations and are formed as surfaces having areas which cause cavitation in the recessed portion 44. With ultrasonic vibrations being transmitted to the cutting portion 34, cavitation is caused at the intersecting surfaces 45a and 45b like the tip 34a of the cutting portion 34. In the recessed portion 44, the cavitation induces a number of bubbles B, and at the same time causes a number of bubbles B to collapse. Therefore, a portion of the small particle of bone 220, which has flowed into the recessed portion 44, is crushed under the action of cavitation, and is progressively changed into still finer small particle of bone 222.

In the recessed portion 44, the volume of cutting fluid rapidly increases by the cavitation. In the recessed portion 44, the pressure hence becomes higher than the pressure between the outer circumferential surface 34b of the cutting portion 34 and a side wall or wall 214 of the bone tunnel 210. Therefore, a portion of the cutting fluid and a portion of the fine small particle of bone 222 in the recessed portion 42 are forced out in a direction as indicated by numeral reference F toward the side wall 214 of the bone tunnel 210 (see FIG. 2C).

Accordingly, the burying portion 40 or recessed portion 44 deflects a flow of a portion of the cutting fluid and a portion of the small particle of bone 220 and 222 between an outer edge 44a of the recessed portion 44 and the side wall 214 of the bone tunnel 210, the side wall 214 opposing the outer edge 44a, in a direction different from the vibrating direction of ultrasonic vibrations. Here, the burying portion 40 deflects the flow of the cutting fluid and the small particle of bone 220 and 222 between the outer edge 44a of the recessed portion 44 and the side wall 214 of the bone tunnel 210, the side wall 214 opposing the outer edge 44a, in a direction that intersects at right angle in the vibrating direction of ultrasonic vibrations. A portion of the cutting fluid and a portion of the small particle of bone 220 and 222 between the side wall 214 of the bone tunnel 210 and the outer circumferential surface 34b of the cutting portion 34 and a portion of the cutting fluid and a portion of the small particle of bone 220 and 222 in the burying portion 40 or recessed portion 44 are therefore caused to flow toward the side wall 214 of the bone tunnel 210.

Similar to as described with respect to the first embodiment, a portion of the small particle of bone 220 and 222, which have been ejected together with the cutting fluid from the recessed portion 44, is therefore buried in the network structure of the side wall 214 of the bone tunnel 210. The recessed portion 44 is formed over the range of at least 360° about the longitudinal axis L. Accordingly, a portion of the small particle of bone 220 and 222 is buried along the shape of the recessed portion 44, in other words, helically in the side wall 214 of the bone tunnel 210.

Here, the bone tunnel 210 is formed to an appropriate depth, so that the cutting portion 34, with ultrasonic vibrations being transmitted thereto, is moved along the direction of the pressing force or the moving direction of the probe 12. The position of the recessed portion 44 relative to the side wall 214 of the bone tunnel 210 therefore develops an offset as the bone tunnel 210 becomes deeper. At this time, the recessed portion 44 continues to bury the small particle of bone 220 and 222 into the side wall 214. The recessed portion 44 is formed over the range of at least 360° about the longitudinal axis L. Accordingly, a part of the side wall 214 is formed as a region with the small particle of bone 220 and 222 buried in in an annular pattern as the bone tunnel 210 becomes deeper.

Depending on the shape of the recessed portion 44, the small particle of bone 220 and 222 are buried and retained over a plurality of times in the side wall 214 of the bone tunnel 210 at a certain location under the action of the recessed portion 44.

Further, the cutting portion 34 in this embodiment has at the proximal end portion thereof a smooth columnar portion 34c in which the recessed portion 44 is not formed. The columnar portion 34c has a contour formed with the same size and shape as the outermost shape-defining portion 36. The recessed portion 44 therefore does not open in a proximal end of the cutting portion 34. Accordingly, a pressure produced by cavitation caused in the recessed portion 44 is prevented from escaping from the inside of the recessed portion 44 toward the proximal end side of the cutting portion 34, so that the pressure rises in the recessed portion 44. The cutting portion 34, with ultrasonic vibrations being transmitted thereto, thus makes the small particle of bone 220 and 222 still finer and facilitates to bury the small particle of bone 220 and 222 in the side wall 214.

The channel width and depth of the recessed portion 44 may be uniform over the entire length thereof. By setting the channel width and depth of the recessed portion 44 to be gradually narrower and shallower from the distal end side toward the proximal end side, the pressure produced by cavitation caused in the recessed portion 44 is caused to rise toward the proximal end side so that the small particle of bone 220 and 222 are made still finer.

### Third Embodiment

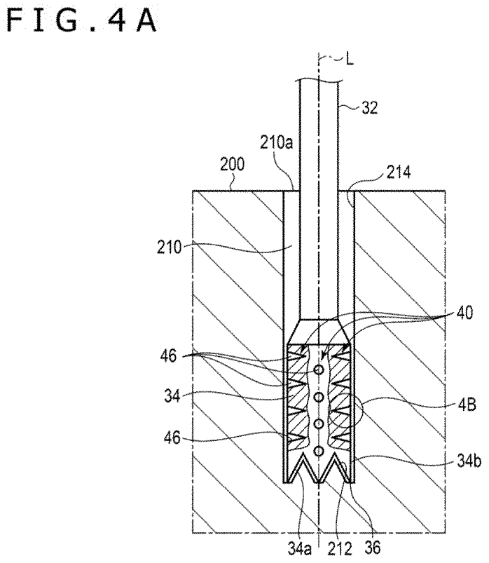

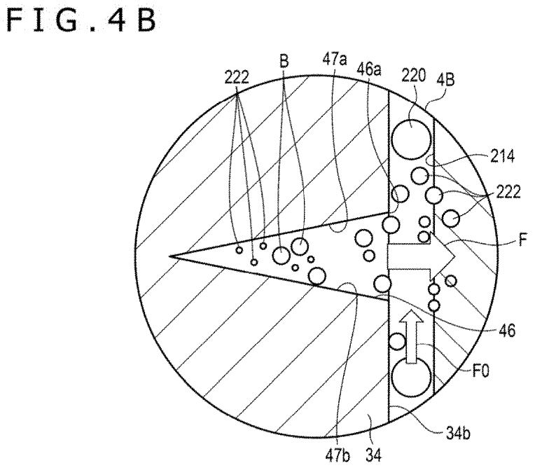

With reference to FIGS. 4A and 4B, a description will next be made about a third embodiment. This embodiment is a modification of the first and second embodiments. As the outer shape of a block-shaped cutting portion 34, desired shapes are allowable without being limited to a cylindrical shape. In an ultrasonic surgical instrument 10 of this embodiment, elements other than the cutting portion 34, such as the probe main body or shaft 32, for example, have similar configurations as in the first and second embodiments described hereinbefore, and therefore their description is omitted herein. In addition, detailed functions of the ultrasonic surgical instrument 10 are also similar to those described hereinbefore with respect to the first and second embodiments, and therefore their description is omitted herein.

FIG. 4A is a view illustrating the external shape of the cutting portion 34 of the ultrasonic surgical instrument 10 according to the third embodiment, and a bone tunnel 210 which is being formed by the cutting portion 34. FIG. 4B is an enlarged schematic view of one of recessed portions 46 disposed in the cutting portion 34 and indicated by numeral reference 4B in FIG. 4A.

A burying portion 40 is disposed in an outer circumferential surface 34b of the cutting portion 34 at a location on the proximal end side of a tip 34a. The burying portion 40 is disposed in the outer circumferential surface 34b of the cutting portion 34 at a location on a proximal end side of a tip of an outermost shape-defining portion 36. Accordingly, the burying portion 40 or recessed portions 46, which will be described hereinafter, are disposed in the outermost shape-defining portion 36.

The burying portion 40 disposed in the cutting portion 34 includes the recessed portions 46 that are conical and extend inward of the cutting portion 34 relative to the outermost shape-defining portion 36. In this embodiment, the recessed portions 46 extend toward a longitudinal axis or central axis L relative to the contour of the outer circumferential surface 34b of the cutting portion 34. The recessed portions 46 are different in shape from the dome-shaped recessed portions 42 described with respect to the first embodiment.

Each recessed portion 46 includes intersecting surfaces or cavitation-causing surfaces 47a and 47b which intersect the vibrating direction of ultrasonic vibrations or an axis parallel to a longitudinal axis L. The intersecting surfaces 47a and 47b in FIG. 4B do not intersect at right angle the longitudinal axis L, but a portion of at least one of the intersecting surfaces 47a and 47b may intersect at right angle the longitudinal axis L. It is therefore suited that the intersecting surfaces 47a and 47b intersect, preferably intersect at right angle the vibrating direction of ultrasonic vibrations and are formed as surfaces having areas which cause cavitation in the recessed portion 46. With ultrasonic vibrations being transmitted to the cutting portion 34, the intersecting surfaces 47a and 47b cause cavitation like the tip 34a of the cutting portion 34.

Similar to as described hereinbefore with respect to the first and second embodiments, a portion of small particle of bone 220, which has flowed into the recessed portion 46, is crushed under the action of cavitation, and is progressively changed into still finer small particle of bone 222. In addition, the pressure in the recessed portion 46 rises owing to the occurrence of the cavitation, so that a portion of the cutting fluid and a portion of the fine small particle of bone 222 in the recessed portion 46 are forced out toward a side wall 214 of the bone tunnel 210 in a direction as indicated by numeral reference F.

Similar to as described hereinbefore with respect to the first embodiment, regions in which the small particle of bone 220 and 222 are buried in a linear pattern parallel to the longitudinal axis L are formed in the side wall 214 of the bone tunnel 210 as the bone tunnel 210 is progressively deepened by the cutting portion 34 with ultrasonic vibrations being transmitted thereto.

As illustrated in FIG. 4A, the tip or a cutting surface 34a of the cutting portion 34 includes a plurality of apex portions or recess portions extending toward the distal end side along the longitudinal axis L. The tip 34a of the cutting portion 34 may be pointed, or may be obtuse. In this embodiment, the bone tunnel 210 includes a bottom surface 212 of a shape different from the example in the first embodiment.

The tip 34a of the cutting portion 34 in this embodiment may be formed like the example (see FIGS. 2A and 2B) described with respect to the first embodiment or the example (see FIG. 3A) described with respect to the second embodiment. This also applies equally to fourth to tenth embodiments.

### Fourth Embodiment

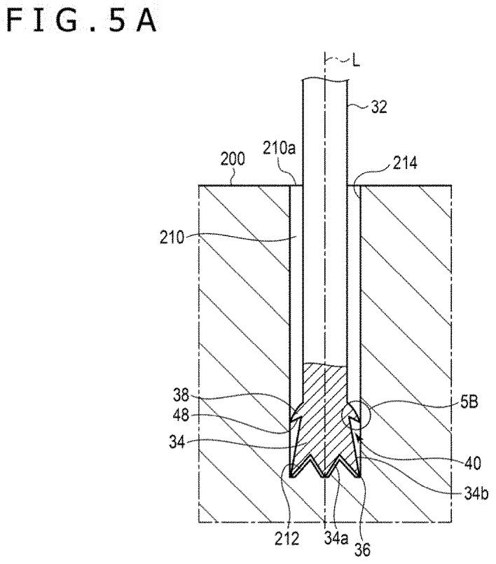

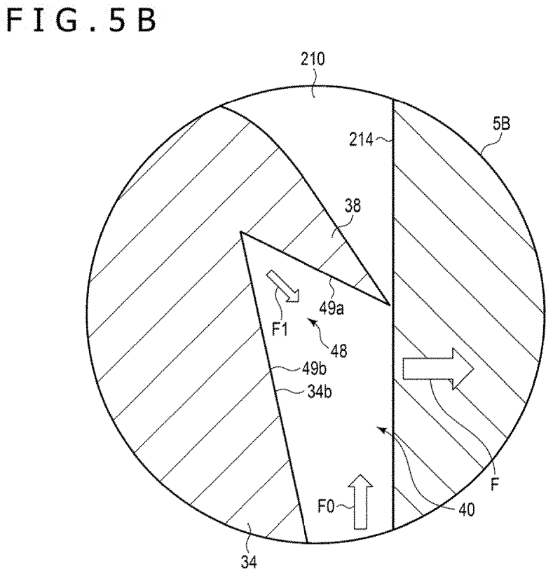

With reference to FIGS. 5A and 5B, a description will next be made about the fourth embodiment. This embodiment is a modification of the first to third embodiments. As the outer shape of a block-shaped cutting portion 34, desired shapes are allowable without being limited to a cylindrical shape. In an ultrasonic surgical instrument 10 of this embodiment, elements other than the cutting portion 34, such as the probe main body or shaft 32, for example, have similar configurations as in the first to third embodiments described hereinbefore, and therefore their description is omitted herein. In addition, detailed functions of the ultrasonic surgical instrument 10 are also similar to those described hereinbefore with respect to the first to third embodiments, and therefore their description is omitted herein.

FIG. 5A is a view illustrating the external shape of the cutting portion 34 of the ultrasonic surgical instrument 10 according to the fourth embodiment, and a bone tunnel 210 which is being formed by the cutting portion 34. FIG. 5B is an enlarged schematic view of a recessed portion 48 disposed in the cutting portion 34 and indicated by numeral reference 5B in FIG. 5A.

An outermost shape-defining portion 36 is disposed on the cutting portion 34 at a location on the distal end side along a longitudinal axis L of a location where a burying portion 40 or the recessed portion 48 is disposed.

The burying portion 40 is disposed in an outer circumferential surface 34b of the cutting portion 34 at a location on the proximal end side of a tip 34a. The burying portion 40 or recessed portions 48, which will be described hereinafter, is disposed in the outer circumferential surface 34b at a location on a proximal end side of the outermost shape-defining portion 36.

The burying portion 40 disposed in the cutting portion 34 includes the recessed portion or a recessed groove 48 that extends inward of the cutting portion 34 relative to the outermost shape-defining portion 36. In this embodiment, the recessed portion 48 extends toward a longitudinal axis or central axis L relative to the contour of the outer circumferential surface 34b of the cutting portion 34. The recessed portion 48 is formed annular.

A flange or protrusion 38 is formed, for example, over a range of 360° on the outer circumferential surface 34b of the cutting portion 34. The flange 38 is formed on the outer circumferential surface 34b of the cutting portion 34 over the entire circumference thereof about the longitudinal axis L. Accordingly, the recessed portion 48 is also formed in the outer circumferential surface 34b of the cutting portion 34 over the entire circumference thereof.

When the proximal end side of the ultrasonic probe 12 is seen from the distal end side thereof, an outermost edge of the flange 38 or a second outermost shape-defining portion is hidden and unseen behind the outermost shape-defining portion or first outermost shape-defining portion 36. When the proximal end side of the ultrasonic probe 12 is seen from the distal end side thereof, the outermost edge of the flange 38 may, however, overlap with the outermost shape-defining portion 36. The outer circumferential surface 34b of the cutting portion 34 therefore inclines so that the outer circumferential surface 34b becomes closer to the longitudinal axis L from the outermost shape-defining portion 36 toward the proximal end side where the flange 38 is formed. Accordingly, the recessed portion 48 is formed in the cutting portion 34 at a location between the outermost shape-defining portion 36 and the flange 38.

The recessed portion 48 includes intersecting surfaces or cavitation-causing surfaces 49a and 49b which intersect the vibrating direction of ultrasonic vibrations or an axis parallel to the longitudinal axis L. The intersecting surface 49a is formed on the flange 38. The intersecting surface 49b is formed between the outermost shape-defining portion 36 and the flange 38.

With ultrasonic vibrations being transmitted, the intersecting surfaces 49a and 49b cause cavitation like the tip 34a of the cutting portion 34. Small particle of bone 220, which has flowed into the recessed portion 48, is therefore changed into still finer small particle of bone 222 under the action of cavitation.

As described hereinbefore, upon occurrence of cavitation at the tip 34a of the cutting portion 34, a flow is induced by the cavitation in the cutting fluid that contains the small particle of bone 220 and 222 and a perfusate. A portion of the cutting fluid is caused to flow together with the crushed small particle of bone 220 in a direction indicated by numeral reference F0, specifically toward the proximal end side of the cutting portion 34 from a gap between the outer circumferential surface 34b of the cutting portion 34 and a side wall 214 of the bone tunnel 210.

In the recessed portion 48 with ultrasonic vibrations being transmitted thereto, a flow is induced in a direction indicated by numeral reference F1 by the shape of the recessed portion 48 and cavitation caused at the intersecting surfaces 49a and 49b in addition to the flow in the direction indicated by numeral reference F0. The flow, which has been induced in the direction indicated by numeral reference F0 by cavitation occurred at the tip 34a of the cutting portion 34 and is going toward an outline of opening 210a of the bone tunnel 210, is decelerated by the flow in the direction indicated by numeral reference F1.

In this embodiment, the extending direction of the intersecting surface 49a is directed toward a bottom surface 212 of the bone tunnel 210 and the longitudinal axis or central axis L rather than a direction intersecting at right angle the longitudinal axis L or a horizontal direction. The intersecting surface 49a therefore causes the cavitation-induced flow to occur in the direction indicated by numeral reference F1 from a side, which is close to the longitudinal axis or central axis L, toward the side of the bottom surface 212 of the bone tunnel 210. The cavitation-induced flow in the direction indicated by numeral reference F0, which has occurred from a distal end side of the cutting portion 34, is then converted by the intersecting surface 49a to a flow directed in a direction indicated by numeral reference F toward the side wall 214 of the bone tunnel 210.

The cutting fluid and the fine small particle of bone 222, which may include the small particle of bone 220 greater in particle size than the small particle of bone 222, in the recessed portion 48 are thus forced out toward the side wall 214 of the bone tunnel 210 as indicated by numeral reference F.

In the side wall 214 of the bone tunnel 210, a portion of the small particle of bone 220 and 222 is progressively buried in an annular pattern.

Accordingly, a region in which the small particle of bone 220 and 222 are buried in the annular pattern about the longitudinal axis L is formed in the side wall 214 of the bone tunnel 210 as the bone tunnel 210 is deepened by the cutting portion 34 with ultrasonic vibrations being transmitted thereto.

### Fifth Embodiment

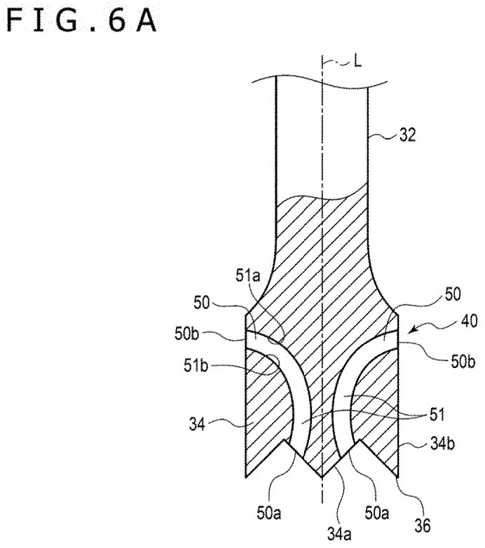

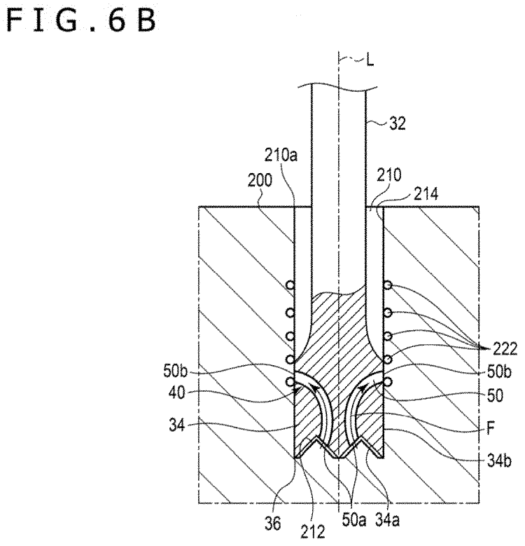

With reference to FIGS. 6A and 6B, a description will next be made about the fifth embodiment. This embodiment is a modification of the first to fourth embodiments. As the outer shape of a block-shaped cutting portion 34, desired shapes are allowable without being limited to a cylindrical shape. In an ultrasonic surgical instrument 10 of this embodiment, elements other than the cutting portion 34, such as the probe main body or shaft 32, for example, have similar configurations as in the first to fourth embodiments described hereinbefore, and therefore their description is omitted herein. In addition, detailed functions of the ultrasonic surgical instrument 10 are also similar to those described hereinbefore with respect to the first to fourth embodiments, and therefore their description is omitted herein.

FIG. 6A is a view illustrating the external shape of the cutting portion 34 of the ultrasonic surgical instrument 10 according to the fifth embodiment. FIG. 6B is a view illustrating the external shape of the cutting portion 34 of the ultrasonic surgical instrument 10, a bone tunnel 210 which is being formed by the cutting portion 34, and a flow of the cutting fluid and small particle of bone as induced by cavitation.

A burying portion 40 is disposed in an outer circumferential surface 34b of the cutting portion 34 at a location on the proximal end side of a tip 34a. The burying portion 40 is disposed in the outer circumferential surface 34b of the cutting portion 34 at a location on the proximal end side of a tip of an outermost shape-defining portion 36. Accordingly, the burying portion 40 or recessed portions 50, which will be described hereinafter, are disposed in the outermost shape-defining portion 36.

The burying portion 40 disposed in the cutting portion 34 includes the recessed portions 50 extending inward of the cutting portion 34 relative to the outermost shape-defining portion 36. In this embodiment, the recessed portions 50 extend toward a longitudinal axis or central axis L relative to the contour of the outer circumferential surface 34b of the cutting portion 34. The recessed portions 50 include channels or through-bores 51, respectively, which communicate the tip 34a and the outer circumferential surface 34b of the cutting portion 34. Continuous channels 51 are therefore formed in the cutting portion 34 so that the cutting channels 51 lead from the cutting surface 34a to the peripheral circumferential surface 34b through the inside of the cutting portion 34. In the tip 34a of the cutting portion 34, first openings 50a are formed as one ends of the channels 51. The first openings 50a hence open toward a bottom surface 212 of the bone tunnel 210. In the outer circumferential surface 34b of the cutting portion 34, second openings 50b are formed as opposite ends of the channels 51. The second openings 50b thus open toward a side wall 214 of the bone tunnel 210.

In this embodiment, the first openings 50a are formed as many as two, while the second openings 50b are formed as many as two or four. This embodiment may include one or more first openings 50a and one or more second openings 50b.

Preferably, each channel 51 is gently curved to include no sharply curved portion. The channel 51 may be formed of a combination of plural straight passages. As an example of the channel 51, a passage at a part continuing to the first opening 50a is parallel to a longitudinal axis L, a passage at a part continuing to the second opening 50b is formed to intersect the longitudinal axis L, and these passages are communicated to each other. The passage continuing to the first opening 50a and the passage continuing to the second opening 50b are arranged at an angle, for example, greater than 90° but equal to or smaller than 180°. The channel 51 may be formed in a substantially Y-shape, substantially T-shape, or the like.

The first opening 50a and the second opening 50b may have the same opening diameter, or different opening diameters. If the opening diameters of the first opening 50a and second opening 50b are different, the channel 51 has an inner diameter that differs between the part closer to the first opening 50a and the part closer to the second opening 50b. For example, the inner diameter of the channel 51 may be formed gradually smaller from the first opening 50a toward the second opening 50b as in that described with respect to the second embodiment. The opening diameters of the first opening 50a and second opening 50b may desirably be greater than the small particle of bone 220 and 222. The opening diameters of the first opening 50a and second opening 50b may desirably be formed, for example, equal to or greater than 50 μm.

Each channel 51 includes, especially at an area close to the second opening 50b, intersecting surfaces or cavitation-causing surfaces 51a and 51b which intersect the vibrating direction of ultrasonic vibrations or an axis parallel to a longitudinal axis L. The intersecting surfaces 51a and 51b do not intersect at right angle the longitudinal axis L, but an area of at least one of the intersecting surfaces 51a and 51b may intersect at right angle the longitudinal axis L. It is therefore suited that the intersecting surfaces 51a and 51b intersect, preferably intersect at right angle the vibrating direction of ultrasonic vibrations and are formed as surfaces having areas which cause cavitation in the recessed portion 50. With ultrasonic vibrations being transmitted to the cutting portion 34, the intersecting surfaces 51a and 51b, with ultrasonic vibrations being transmitted to the cutting portion 34, cause cavitation like the tip 34a of the cutting portion 34.

Upon occurrence of cavitation at the tip 34a of the cutting portion 34, a flow is induced by the cavitation in the cutting fluid. A portion of the cutting fluid is caused to flow together with crushed small particle of bone 220 toward the proximal end side of the cutting portion 34 from a gap between the outer circumferential surface 34b of the cutting portion 34 and the side wall 214 of the bone tunnel 210. In addition, a portion of the cutting fluid is forced out, together with crushed small particle of bone 220, from the first opening 50a toward the second opening 50b of the recessed portion 50. A portion of the cutting fluid and a portion of the crushed small particle of bone 220 are hence forced out toward the proximal end side of the cutting portion 34. Similar to as described hereinbefore with respect to the first to fourth embodiments, a portion of small particle of bone 220, which has flowed into the recessed portion 50, is crushed under the action of cavitation, and is progressively changed into still finer small particle of bone 222.

Further, bubbles, which have been induced by the cavitation caused at the intersecting surfaces 51a and 51b, are also forced out from the first opening 50a toward the second opening 50b of the recessed portion 50.

Accordingly, the burying portion 40 or each recessed portion 50 deflects a flow of a portion of the cutting fluid and a portion of the small particle of bone 220 and 222 in the recessed portion 50 and between the second opening 50b and the side wall 214 of the bone tunnel 210, the side wall 214 opposing the second opening 50b, in a direction different from the vibrating direction of ultrasonic vibrations. A portion of the cutting fluid and a portion of the small particle of bone 220 and 222 between the side wall 214 of the bone tunnel 210 and the outer circumferential surface 34b of the cutting portion 34 and a portion of the cutting fluid and a portion of the small particle of bone 220 and 222 in the burying portion 40 or the recessed portion 50 are therefore caused to flow toward the side wall 214 of the bone tunnel 210.

Accordingly, a portion of the small particle of bone 220 and 222, which has been ejected together with the cutting fluid from the recessed portion 50 through the second opening 50b, is buried in the network structure of the side wall 214 of the bone tunnel 210.

Accordingly, a region in which the small particle of bone 220 and 222 are buried in a linear pattern parallel to the longitudinal axis L is formed in the side wall 214 of the bone tunnel 210 as the bone tunnel 210 is deepened by the cutting portion 34 with ultrasonic vibrations being transmitted thereto.

### Sixth Embodiment

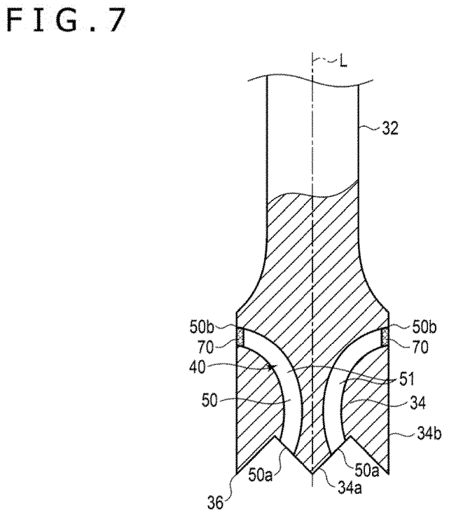

Using FIG. 7, a description will next be made about the sixth embodiment. This embodiment is a modification of the fifth embodiment.

FIG. 7 is a view illustrating the external shape of a cutting portion 34 of an ultrasonic surgical instrument 10 according to the sixth embodiment.

In the second opening 50b of each recessed portion 50, a filter 70 is disposed. Here, a porous Ti filter, for example, is used as the filter 70. As an example of a method of forming a porous Ti layer, the technique disclosed, for example, in U.S. Pat. No. 5,843,289 entitled “Surface Modification of Medical Implants” may be used.

In this embodiment, a portion of small particle of bone 220 moves together with the cutting fluid from the first opening 50a toward the second opening 50b of the recessed portion 50 as described with respect to the fifth embodiment. Along with the cutting fluid, the portion of the small particle of bone 220 therefore hits the filter 70 at high speed. Accordingly, the filter 70 finely crushes the small particle of bone 220, in other words, comminutes the small particle of bone 220. The filter 70 then discharges the small particle of bone 222 from the second opening 50b toward the side wall 214 of the bone tunnel 210.

Similar to as described with respect to the fifth embodiment, a portion of the small particle of bone 220 and 222, which has been ejected together with the cutting fluid from the recessed portion 50, is hence buried in the network structure of the side wall 214 of the bone tunnel 210.

Accordingly, a region in which the small particle of bone 220 and 222 are buried in a linear pattern parallel to the longitudinal axis L is formed in the side wall 214 of the bone tunnel 210 as the bone tunnel 210 is deepened by the cutting portion 34 with ultrasonic vibrations being transmitted thereto.

### Seventh Embodiment

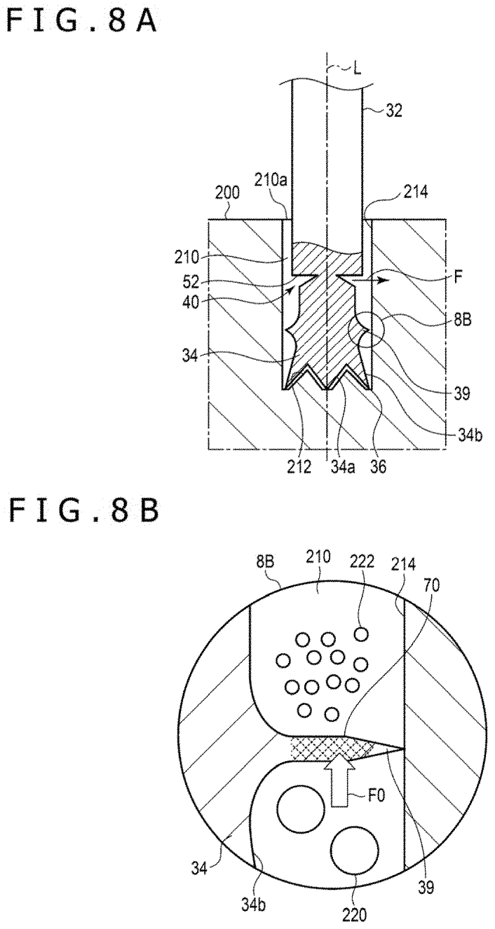

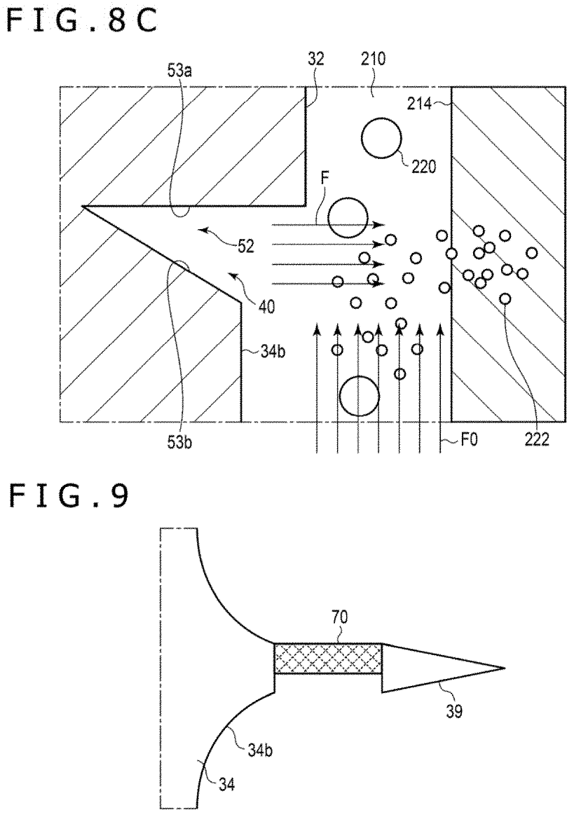

With reference to FIGS. 8A to 8C, a description will next be made about the seventh embodiment. This embodiment is a modification of the first to sixth embodiments. As the outer shape of a block-shaped cutting portion 34, desired shapes are allowable without being limited to a cylindrical shape. In an ultrasonic surgical instrument 10 of this embodiment, elements other than the cutting portion 34, such as the probe main body or shaft 32, for example, have similar configurations as in the first to sixth embodiments described hereinbefore, and therefore their description is omitted herein. In addition, detailed functions of the ultrasonic surgical instrument 10 are also similar to those described hereinbefore with respect to the first to sixth embodiments, and therefore their description is omitted herein.

FIG. 8A is a view illustrating the external shape of the cutting portion 34 of the ultrasonic surgical instrument 10 according to the seventh embodiment and a bone tunnel 210 which is being formed by the cutting portion 34. FIG. 8B is an enlarged schematic view of a flange 39 disposed on the cutting portion 34 as indicated by numeral reference 8B in FIG. 8A. FIG. 8C is a view illustrating a recessed portion 52 disposed in the cutting portion 34 and a flow of the cutting fluid and small particle of bone as induced by cavitation.

An outermost shape-defining portion 36 is disposed on the cutting portion 34 at a location on the distal end side along a longitudinal axis L of a location where a burying portion 40 or the recessed portion 52 is disposed.

The burying portion 40 is disposed in an outer circumferential surface 34b of the cutting portion 34 at a location on the proximal end side of a tip 34a. In other words, the burying portion 40 or the recessed portion 52, which will be described hereinafter, is disposed in the outer circumferential surface 34b at a location on the proximal end side of the outermost shape-defining portion 36.

The burying portion 40 disposed in the cutting portion 34 includes the recessed portion or a recessed groove 52 that extends inward of the cutting portion 34 relative to the outermost shape-defining portion 36. In this embodiment, the recessed portion 52 extends toward a longitudinal axis or central axis L relative to the contour of the outer circumferential surface 34b of the cutting portion 34. The recessed portion 52 is formed annular.

The flange 39 is formed, for example, a range of 360° on the outer circumferential surface 34b of the cutting portion 34 between the outermost shape-defining portion 36 and the recessed portion 52. The flange 39 is formed on the outer circumferential surface 34b of the cutting portion 34 over the entire circumference thereof about the longitudinal axis L.

When the proximal end side of the ultrasonic probe 12 is seen from the distal end side thereof, an outermost edge of the flange 39 or a second outermost shape-defining portion is hidden and unseen behind the outermost shape-defining portion or first outermost shape-defining portion 36. When the proximal end side of the ultrasonic probe 12 is seen from the distal end side thereof, the outermost edge of the flange 39 may, however, overlap with the outermost shape-defining portion 36. The outer circumferential surface 34b of the cutting portion 34 therefore inclines so that the outer circumferential surface 34b becomes closer to the longitudinal axis L from the outermost shape-defining portion 36 toward the proximal end side where the flange 39 is formed.

For example, the flange 39 is made from bulk titanium alloy, and has been made porous by plasma etching processing. A filter 70 is hence formed as a porous Ti layer in the flange 39.

The recessed portion 52 is disposed between the flange 39 and a tip of the probe main body or shaft 32. The recessed portion 52 includes intersecting surfaces or cavitation-causing surfaces 53a and 53b which intersect the vibrating direction of ultrasonic vibrations or the axis parallel to the longitudinal axis L. The intersecting surface 53a intersects at right angle in the vibrating direction of ultrasonic vibrations to be transmitted to the cutting portion 34. It is therefore suited that the intersecting surface 53a intersects, preferably intersects at right angle in the vibrating direction of ultrasonic vibrations and is formed as a surface having an area which causes cavitation in the recessed portion 52. A description will hereinafter be made about an example in which the intersecting surface 53a intersects at right angle in the vibrating direction of ultrasonic vibrations, although it is also suited that the intersecting surface 53b intersects at right angle in the vibrating direction of ultrasonic vibrations and is formed as a surface having an area which causes cavitation in the recessed portion 52. Accordingly, some parts or the entire parts of both the intersecting surfaces 53a and 53b may intersect the vibrating direction of ultrasonic vibrations at right angle, or a part or the entire part of one of the intersecting surfaces 53a and 53b may intersect the vibrating direction of ultrasonic vibrations at right angle.

Upon occurrence of cavitation at the tip 34a of the cutting portion 34, a flow is induced by the cavitation in the cutting fluid as described hereinbefore. A portion of the cutting fluid is caused to flow together with crushed small particle of bone 220 in a direction indicated by numeral reference F0 toward the proximal end side of the cutting portion 34 from a gap between the outer circumferential surface 34b of the cutting portion 34 and a side wall 214 of the bone tunnel 210.

The cutting fluid and small particle of bone 220 go toward an outline of opening 210a of the bone tunnel 210 through the filter 70. Described specifically, the small particle of bone 220 moves along the longitudinal axis L toward the proximal end side between the outer circumferential surface 34b of the cutting portion 34 and the side wall 214 of the bone tunnel 210, and hits the filter 70 at high speed. Accordingly, the filter 70 finely crushes the small particle of bone 220, in other words, comminutes the small particle of bone 220. The filter 70 then discharges the small particle of bone 222 toward the outline of opening 210a of the bone tunnel 210.

As described hereinbefore, this embodiment enables to discharge small particle of bone 222 of reduced particle size toward the outline of opening 210a of the bone tunnel 210 by passing small particle of bone 220 of large particle size produced at the cutting portion 34 through the physical porous filter 70.

Similar to the tip 34a of the cutting portion 34, the intersecting surfaces 53a and 53b of the recessed portion 52, with ultrasonic vibrations being transmitted thereto, cause cavitation. The small particle of bone 220, which has flowed into the recessed portion 52, is hence changed into still finer small particle of bone 222 under the action of the cavitation.

In the recessed portion 52 with ultrasonic vibrations being transmitted thereto, the pressure becomes higher than the pressure between the outer circumferential surface 34b of the cutting portion 34 and the side wall or wall 214 of the bone tunnel 210 owing to the cavitation caused at the intersecting surfaces 53a and 53b. The cutting fluid and the fine small particle of bone 222 in the recessed portion 52 are therefore forced out in a direction as indicated by numeral reference F toward the side wall 214 of the bone tunnel 210.

Accordingly, the burying portion 40 deflects a flow of the cutting fluid in a vicinity of the recessed portion 52 in a direction different from the vibrating direction of ultrasonic vibrations. Described specifically, the burying portion 40 deflects a flow of the cutting fluid and small particle of bone 220 and 222 in the vicinity of the recessed portion 52 in the direction different from the vibrating direction of ultrasonic vibrations.

As described hereinbefore, the recessed portion 52 disposed in the cutting portion 34, to which ultrasonic vibrations are being transmitted, directs a flow of the cutting fluid, which contains the small particle of bone 220 and 222 produced upon formation of the bone tunnel 210, toward the side wall 214 of the bone tunnel 210 by the ultrasonic vibrations.

In the side wall 214 of the bone tunnel 210, a portion of the small particle of bone 220 and 222 is therefore progressively buried in an annular pattern.

Accordingly, a region in which the small particle of bone 220 and 222 are buried in the annular pattern about the longitudinal axis L is formed in the side wall 214 of the bone tunnel 210 as the bone tunnel 210 is deepened by the cutting portion 34 with ultrasonic vibrations being transmitted thereto.

As a modification, the filter 70 may be disposed in a state illustrated in FIG. 9. A filter 70 illustrated in FIG. 9 is produced separately from the flange 39, and is embedded in the flange 39 in a post-step.

As the filter 70 illustrated by way of example in FIG. 8B, the description was made about the example in which the filter 70 was formed in the flange 39 by plasma etching processing. As depicted in FIG. 9, a porous Ti filter 70 may be embedded in the flange 39 of the cutting portion 34.

### Eighth Embodiment

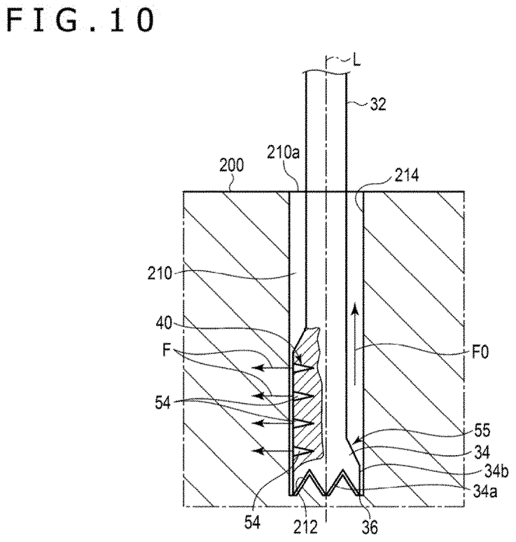

With reference to FIG. 10, a description will next be made about the eighth embodiment. This embodiment is a modification of the first to seventh embodiments. As the outer shape of a block-shaped cutting portion 34, desired shapes are allowable without being limited to a cylindrical shape. In an ultrasonic surgical instrument 10 of this embodiment, elements other than the cutting portion 34, such as the probe main body or shaft 32, for example, have similar configurations as in the first to seventh embodiments described hereinbefore, and therefore their description is omitted herein. In addition, detailed functions of the ultrasonic surgical instrument 10 are also similar to those described hereinbefore with respect to the first to seventh embodiments, and therefore their description is omitted herein.

FIG. 10 is a view illustrating the external shape of the cutting portion 34 of the ultrasonic surgical instrument 10 according to the eighth embodiment and a bone tunnel 210 which is being formed by the cutting portion 34.

An outermost shape-defining portion 36 is disposed on the cutting portion 34 at a location on the distal end side along a longitudinal axis L of a location where a burying portion 40 or recessed portions 54 are disposed.

The cutting portion 34 in this embodiment is formed asymmetrical with respect to the longitudinal axis L.

The burying portion 40 is disposed in an outer circumferential surface 34b of the cutting portion 34 at a location on the proximal end side of a tip 34a. The burying portion 40 is disposed in the outer circumferential surface 34b of the cutting portion 34 at a location on the proximal end side of a tip of an outermost shape-defining portion 36. Accordingly, the burying portion 40 or recessed portions 54, which will be described hereinafter, are disposed in the outermost shape-defining portion 36.

The burying portion 40 disposed in the cutting portion 34 includes a plurality of recessed conical portions 54 that extend inward of the cutting portion 34 relative to the outermost shape-defining portion 36. In this embodiment, the recessed portions 54 extend toward the longitudinal axis or central axis L relative to the contour of the outer circumferential surface 34b of the cutting portion 34. Each recessed portion 54 is formed similar to the recessed portions 46 described with respect to the third embodiment. In this embodiment, the recessed portions 54 are arranged in a row along the longitudinal axis L in the outer circumferential surface 34b of the cutting portion 34.

Described specifically, the recessed conical portions 54 similar to the recessed portions 46 described with respect to the third embodiment are formed in a part of the outer circumferential surface 34b of the cutting portion 34, for example, over a half of the circumference of the outer circumferential surface 34b of the cutting portion 34.

Similar to the recessed portions 46 described with respect to the third embodiment, small particle of bone 220 and 222 can hence be buried by the recessed portions 54 in an opposing side wall 214 of the bone tunnel 210. Accordingly, a region in which the small particle of bone 220 and 222 are buried in a linear pattern parallel to the longitudinal axis L is formed in the side wall 214 of the bone tunnel 210 as the bone tunnel 210 is deepened by the cutting portion 34 with ultrasonic vibrations being transmitted thereto.

In the remaining, substantially half on the opposite side of the circumference of the outer circumferential surface 34b of the cutting portion 34, a single recessed portion 55 is formed extending inward of the cutting portion 34 relative to the outermost shape-defining portion 36 of the cutting portion 34. A part between the tip of the probe main body (shaft) 32 and the recessed portion 55 in a proximal end of the cutting portion 34 is formed parallel to the longitudinal axis L, and no recess-protrusion configuration is formed there. Unlike the recessed portions 54, no flow is hence formed in the recessed portion 55 to force out the cutting fluid and small particle of bone 220 and 222 toward the side wall 214 of the bone tunnel 210. In this embodiment, a flow induced by cavitation caused at the tip 34a of the cutting portion 34 goes toward an outline of opening 210a of the bone tunnel 210 through a gap between the recessed portion 55 and the side wall 214 of the bone tunnel 210, whereby the bone tunnel 210 is efficiently formed.

By asymmetrically forming the cutting portion 34 with respect to the longitudinal axis L, the small particle of bone 220 and 222 can be introduced into the side wall 214 of the bone tunnel 210 at only one or more desired areas thereof.

It is possible, for example, to positively bury the small particle of bone 220 and 222 only in a direction, in which loads tend to be applied by knee bending and stretching actions, to promote healing, and to avoid positive introduction of the small particle of bone 220 and 222 in other directions.

### Ninth Embodiment

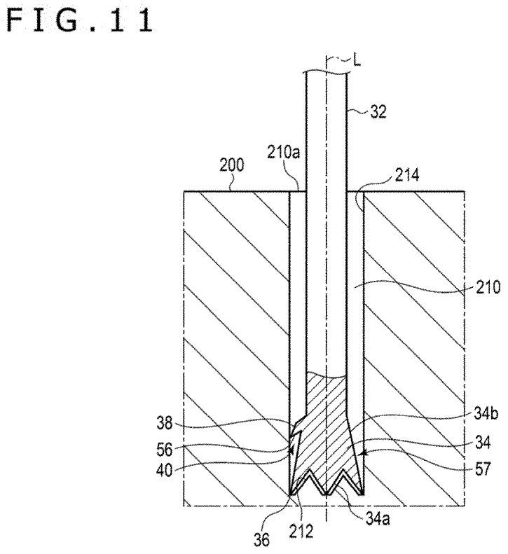

With reference to FIG. 11, a description will next be made about the ninth embodiment. This embodiment is a modification of the first to eighth embodiments. As the outer shape of a block-shaped cutting portion 34, desired shapes are allowable without being limited to a cylindrical shape. In an ultrasonic surgical instrument 10 of this embodiment, elements other than the cutting portion 34, such as the probe main body or shaft 32, for example, have similar configurations as in the first to eighth embodiments described hereinbefore, and therefore their description is omitted herein. In addition, detailed functions of the ultrasonic surgical instrument 10 are also similar to those described hereinbefore with respect to the first to eighth embodiments, and therefore their description is omitted herein.

FIG. 11 is a view illustrating the external shape of the cutting portion 34 of the ultrasonic surgical instrument 10 according to the ninth embodiment and a bone tunnel 210 which is being formed by the cutting portion 34.

An outermost shape-defining portion 36 is disposed on the cutting portion 34 at a location on the distal end side along a longitudinal axis L of a location where a burying portion 40 or recessed portion 56 is disposed.

The cutting portion 34 in this embodiment is formed asymmetrical with respect to the longitudinal axis L.

The burying portion 40 is disposed in an outer circumferential surface 34b of the cutting portion 34 at a location on the proximal end side of a tip 34a. The burying portion 40 or the recessed portion 56, which will be described hereinafter, is disposed in the outer circumferential surface 34b of the cutting portion 34 at a location on the proximal end side of the outermost shape-defining portion 36.

The burying portion 40 disposed in the cutting portion 34 includes a recessed portion 56 that extends inward of the cutting portion 34 relative to the outermost shape-defining portion 36. In this embodiment, the recessed portion 56 extends toward the longitudinal axis or central axis L relative to the contour of the outer circumferential surface 34b of the cutting portion 34. The recessed portion 56 is formed similar to the recessed portion 48 described with respect to the fourth embodiment.

Described specifically, the recessed portion 56 similar to the recessed portion 48 described with respect to the fourth embodiment is formed in a part of the outer circumferential surface 34b of the cutting portion 34, for example, over a half of the circumference of the outer circumferential surface 34b of the cutting portion 34. A flange or burr 38 is formed, for example, over a range of approximately 180° about the longitudinal axis L on the outer circumferential surface 34b of the cutting portion 34. The flange or burr 38 is therefore formed on the cutting portion 34 by the recessed portion 56. When the proximal end side of the ultrasonic probe 12 is seen from the distal end side thereof, the flange 38 is hidden and unseen behind the outermost shape-defining portion 36. When the proximal end side of the ultrasonic probe 12 is seen from the distal end side thereof, the outermost edge of the flange 38 may, however, overlap with the outermost shape-defining portion 36.

The flange 38 is formed on the outer circumferential surface 34b of the cutting portion 34 over approximately 180° about the longitudinal axis L. Accordingly, the recessed portion 56 is also formed over approximately 180° about the longitudinal axis L in the outer circumferential surface 34b of the cutting portion 34.

Similar to the recessed portion 48 described with respect to the fourth embodiment, small particle of bone 220 and 222 can hence be buried by the recessed portion 56 in an opposing side wall 214 of the bone tunnel 210. Accordingly, a region in which the small particle of bone 220 and 222 are buried in a half-pipe pattern parallel to the longitudinal axis L is formed in the side wall 214 of the bone tunnel 210 as the bone tunnel 210 is deepened by the cutting portion 34 with ultrasonic vibrations being transmitted thereto.

In the remaining, substantially half on the opposite side of the circumference of the outer circumferential surface 34b of the cutting portion 34, a single recessed portion 57 is formed extending inward of the cutting portion 34 relative to the outermost shape-defining portion 36 of the cutting portion 34. A part between the tip of the probe main body or shaft 32 and the recessed portion 57 in a proximal end of the cutting portion 34 is formed parallel to the longitudinal axis L, and no recess-protrusion configuration is formed there. Unlike the recessed portion 56, no flow is hence formed in the recessed portion 57 to force out the cutting fluid and small particle of bone 220 and 222 toward the side wall 214 of the bone tunnel 210. In this embodiment, a flow induced by cavitation caused at the tip 34a of the cutting portion 34 goes toward an outline of opening 210a of the bone tunnel 210 through a gap between the recessed portion 57 and the side wall 214 of the bone tunnel 210, whereby the bone tunnel 210 is efficiently formed.

By asymmetrically forming the cutting portion 34 with respect to the longitudinal axis L, the small particle of bone 220 and 222 can be introduced into the side wall 214 of the bone tunnel 210 at only one or more desired areas thereof.

It is possible, for example, to positively bury the small particle of bone 220 and 222 only in a direction, in which loads tend to be applied by knee bending and stretching actions, to promote healing, and to avoid positive introduction of the small particle of bone 220 and 222 in other directions.

### Tenth Embodiment

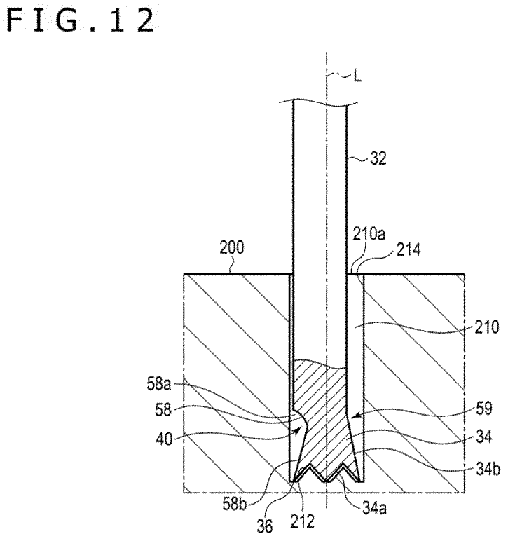

With reference to FIG. 12, a description will next be made about the tenth embodiment. This embodiment is a modification of the first to ninth embodiments. As the outer shape of a block-shaped cutting portion 34, desired shapes are allowable without being limited to a cylindrical shape. In an ultrasonic surgical instrument 10 of this embodiment, elements other than the cutting portion 34, such as the probe main body or shaft 32, for example, have similar configurations as in the first to ninth embodiments described hereinbefore, and therefore their description is omitted herein. In addition, detailed functions of the ultrasonic surgical instrument 10 are also similar to those described hereinbefore with respect to the first to ninth embodiments, and therefore their description is omitted herein.

FIG. 12 is a view illustrating the external shape of the cutting portion 34 of the ultrasonic surgical instrument 10 according to the tenth embodiment and a bone tunnel 210 which is being formed by the cutting portion 34.

An outermost shape-defining portion 36 is disposed on the cutting portion 34 at a location on the distal end side along a longitudinal axis L of a location where a burying portion 40 or recessed portion 58 is disposed.

The cutting portion 34 in this embodiment is formed asymmetrical with respect to the longitudinal axis L.

The burying portion 40 is disposed in an outer circumferential surface 34b of the cutting portion 34 at a location on the proximal end side of a tip 34a. The burying portion 40 or the recessed portion 58, which will be described hereinafter, is disposed in the outer circumferential surface 34b at a location on the proximal end side of an outermost shape-defining portion 36.

The burying portion 40 disposed in the cutting portion 34 includes the recessed portion or a notch 58 that extends inward of the cutting portion 34 relative to the outermost shape-defining portion 36. In this embodiment, the recessed portion 58 extends toward the longitudinal axis or a central axis L relative to the contour of the outer circumferential surface 34b of the cutting portion 34. The recessed portion 58 is formed over a range of, for example, 180° about the longitudinal axis L.

In this embodiment, a region which is indicated by numeral reference 58a and extends from a bottom portion of the recessed portion 58 toward the proximal end side of the cutting portion 34 is formed in a curved state. A region which is indicated by numeral reference 58b and extends from the bottom portion of the recessed portion 58 toward the tip 34a of the cutting portion 34 is formed linear. These regions indicated by numeral references 58a and 58b can be used as intersecting surfaces that intersect the vibrating direction of ultrasonic vibrations. With ultrasonic vibrations being transmitted to the cutting portion 34, cavitation is therefore caused at the intersecting surfaces 58a and 58b like the tip 34a of the cutting portion 34. In the recessed portion 58, the cavitation induces a number of bubbles B (see, for example, FIG. 2C), and at the same time causes a number of bubbles B to collapse. Therefore, a portion of small particle of bone 220, which has flowed into the recessed portion 58, is crushed under the action of cavitation, and is progressively changed into still finer small particle of bone 222.

In the recessed portion 58, the volume of cutting fluid rapidly increases by the cavitation. Similar to as described with respect to the first to ninth embodiments, a portion of the cutting fluid and a portion of the small particle of bone 220 and 222 in the burying portion 40 or the recessed portion 58 are therefore caused to flow toward a side wall 214 of the bone tunnel 210.

In the side wall 214 of the bone tunnel 210, a portion of the small particle of bone 220 and 222 is therefore progressively buried in a half-pipe pattern.

Accordingly, a region in which the small particle of bone 220 and 222 are buried in the half-pipe pattern about the longitudinal axis L is formed in the side wall 214 of the bone tunnel 210 as the bone tunnel 210 is deepened by the cutting portion 34 with ultrasonic vibrations being transmitted thereto.

In the remaining, substantially half on the opposite side of the circumference of the outer circumferential surface 34b of the cutting portion 34, a single recessed portion 59 is formed extending inward of the cutting portion 34 relative to the outermost shape-defining portion 36 of the cutting portion 34. A part between the tip of the probe main body or shaft 32 and the recessed portion 59 in a proximal end of the cutting portion 34 is formed parallel to the longitudinal axis L, and no recess-protrusion configuration is formed there.

In the recessed portion 59, a region, which is symmetrical with the region indicated by numeral reference 58b with respect to the longitudinal axis L, can cause cavitation a little with ultrasonic vibrations being transmitted thereto. However, this region is nearly in the state of a disposition parallel to the longitudinal axis L so that the intensity of cavitation to be caused is significantly smaller compared with the intensity of cavitation to be caused at the region indicated by numeral reference 58a. In the recessed portion 59, a flow is hence not formed to such an energy as in the recessed portion 58 to force out the cutting fluid and small particle of bone 220 and 222 toward the side wall 214 of the bone tunnel 210. In this embodiment, a flow induced by cavitation caused at the tip 34a of the cutting portion 34 goes toward an outline of opening 210a of the bone tunnel 210 through between the recessed portion 59 and the side wall 214 of the bone tunnel 210, whereby the bone tunnel 210 is efficiently formed.

By asymmetrically forming the cutting portion 34 with respect to the longitudinal axis L, the small particle of bone 220 and 222 can be introduced into the side wall 214 of the bone tunnel 210 at only one or more desired areas thereof.

It is possible, for example, to positively bury the small particle of bone 220 and 222 only in a direction, in which loads tend to be applied by knee bending and stretching actions, to promote healing, and to avoid positive introduction of the small particle of bone 220 and 222 in other directions.

### Comparative Example

The formation of a bone tunnel by the ultrasonic probe 12 of each embodiment described hereinbefore has the following technical differences compared with the formation of a bone tunnel by an undepicted conventional drill.

The formation of the bone tunnel by the ultrasonic probe 12 can form, in conformity with the shape of a tendon graft, a bone tunnel 210 of a desired shape corresponding to the shape of the ultrasonic probe 12 as projected in its axial direction. In addition, the ultrasonic probe 12 can reduce the gap between the bone tunnel 210 and the tendon graft, and therefore can expect early healing and is clinically effective.

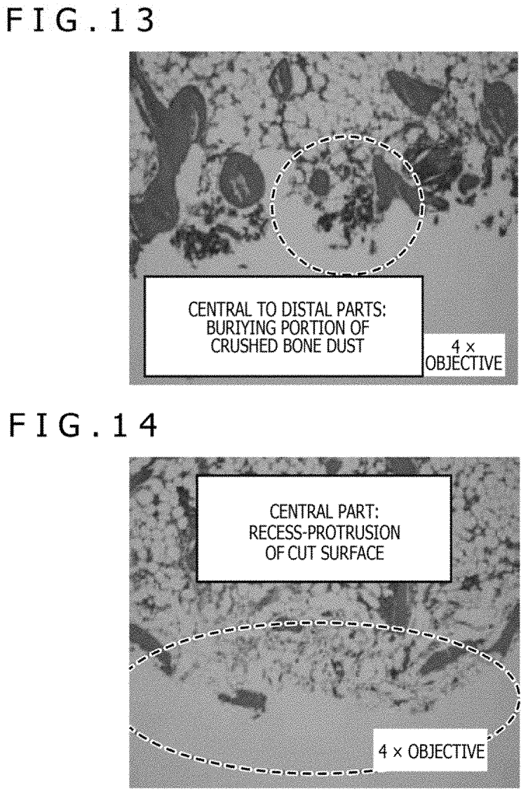

Through a pathological evaluation based on a comparison between FIG. 13, which depicts a bone tunnel 210 formed by the ultrasonic probe 12, and FIG. 14, which depicts a bone tunnel formed by the drill, it has been found that, when a bone tunnel 210 is formed by the ultrasonic probe 12, small particle of bone 220 and 222 of various particle sizes are buried in the side wall 214 of the bone tunnel 210. Described specifically, in FIG. 13, crushed small particle of bone 220 and 222 are buried from a central part to a distal part in the figure. In FIG. 14, on the other hand, it can be seen that a recess-protrusion configuration remains at a cut surface in a central part and no crushed small particle of bone are buried there.

In the example in which the drill was used, the example being shown in FIG. 14, the recess-protrusion configuration remains at the cut surface in the central part. Compared with the formation of the bone tunnel 210 by the ultrasonic probe 12, the side wall of the bore tunnel is formed rough. In the example in which the ultrasonic probe 12 was used, the example being depicted in FIG. 13, on the other hand, only a slightest recess-protrusion configuration remains, and the side wall 214 of the bone tunnel 210 is formed smoother compared with the formation by the drill. Concerning the formation of the bone tunnel 210, the use of the ultrasonic probe 12, with ultrasonic vibrations being transmitted thereto, therefore forms the side wall 214 smoother compared with the use of the drill. As to dusts 220 and 222, on the other hand, the use of the ultrasonic probe 12, with ultrasonic vibrations being transmitted thereto, buries them more in the side wall 214 of the bone tunnel 210 compared with the use of the drill.

The disclosed technology is not limited to the embodiments described hereinbefore, and various modifications are possible in practice within a scope not departing from the spirit of the disclosed technology. Further, the individual embodiments may be practiced in combination as much as possible as needed, and in this case, combined advantageous effects can be brought about. Furthermore, inventions of various levels are included in the embodiments described hereinbefore, and a variety of inventions can be derived by appropriate combinations of the plural features disclosed herein.

In sum, one aspect of the disclosed technology is directed to an ultrasonic surgical instrument comprising an elongated shaft having respective proximal and distal end sides. An ultrasonic transducer is secured to the shaft via the proximal end side. The ultrasonic transducer configured to generate and to transmit ultrasonic vibrations from the proximal end side toward a distal end side along a longitudinal axis of the shaft. A cutting portion is disposed on the distal end side of the shaft. The cutting portion includes an outermost shape-defining portion that defines an outermost shape having a block shape that when pressed at a tip of the block shape against a bone while in a fluid with the ultrasonic vibrations being transmitted thereto, cuts the bone in a direction of a pressing force to form a bone tunnel and to produce small particle of bone at the tip. A burying portion is disposed on the proximal end side of the outermost shape-defining portion and is configured to direct a flow of the fluid, which contains the small particle of bone produced upon formation of the bone tunnel, toward a wall formed in the bone tunnel by the cutting portion along the direction of the pressing force and to bury the small particle of bone in the wall of the bone tunnel.

The tip of the cutting portion while the ultrasonic vibrations is transmitted, induces the flow of the fluid in a direction along a vibrating direction of the ultrasonic vibrations. The burying portion deflects the flow of the fluid in a direction different from the vibrating direction of the ultrasonic vibrations. The burying portion is disposed on an outer circumferential surface of the cutting portion at a location on the proximal end side. The burying portion is disposed on an outer circumferential surface of the cutting portion at a location on the proximal end side of the outermost shape-defining portion.

The burying portion has one or more recessed portions extending inward of the cutting portion relative to the outermost shape-defining portion and is configured to direct the flow of the fluid, which contains the small particle of bone produced upon formation of the bone tunnel by the ultrasonic vibrations toward the wall formed by the outermost shape-defining portion. The one or more recessed portions have a surface area that intersects the vibrating direction of the ultrasonic vibrations and induces cavitation in the one or more recessed portions. The surface area intersects the vibrating direction at a right angle at the area that induces the cavitation. The one or more recessed portions include a channel communicating between the one or more recessed portion and the tip of the cutting portion.

The channel includes a first opening that opens toward a bottom surface of the bone tunnel, a second opening that opens toward the wall and a channel surface that intersects the vibrating direction of the ultrasonic vibrations. The one or more recessed portions include an outer edge opposing the wall formed in the bone tunnel. The outer edge is formed with an outer shape and size so that a portion of the small particle of bone is produced upon formation of the bone tunnel is received in the one or more recessed portions. The recessed portions are arranged along the direction of the pressing force. The cutting portion is configured to form the bone tunnel with an opening having a shape corresponding to a shape of the outermost shape-defining portion. The cutting portion includes a tip surface that intersects the vibrating direction of the ultrasonic vibrations and wherein the tip surface is configured to cause cavitation to induce a flow in the fluid. The ultrasonic surgical instrument further includes a filter disposed in the cutting portion and configured to permit the small particle of bone produced upon formation of the bone tunnel to pass through the filter together with the flow of the fluid induced by the cavitation. The outermost shape-defining portion with the ultrasonic vibrations is transmitted to the cutting portion forming the wall in the bone tunnel.

Another aspect of the disclosed technology is directed to an ultrasonic surgical assembly comprising an ultrasonic surgical instrument having an elongated shaft including respective proximal and distal end sides. An ultrasonic transducer is secured to the shaft via the proximal end side. The ultrasonic transducer is configured to generate and to transmit ultrasonic vibrations from the proximal end side toward a distal end side along a longitudinal axis of the shaft. A cutting portion is disposed on the distal end side of the shaft. The cutting portion includes an outermost shape-defining portion that defines an outermost shape having a block shape that when pressed at a tip of the block shape against a bone while in a fluid with the ultrasonic vibrations being transmitted thereto, cuts the bone in a direction of a pressing force to form a bone tunnel and to produce small particle of bone at the tip. A burying portion is disposed on the proximal end side of the outermost shape-defining portion and is configured to direct a flow of the fluid, which contains the small particle of bone produced upon formation of the bone tunnel toward a wall formed in the bone tunnel by the cutting portion along the direction of the pressing force and to bury the small particle of bone in the wall of the bone tunnel. An ultrasonic vibration generating section is attached on a proximal end side of the ultrasonic surgical instrument.

A further aspect of the disclosed technology is directed to a method of operating an ultrasonic surgical instrument for forming a bone tunnel using an ultrasonic device having a shaft. The shaft includes a distal end and a proximal end and a treatment portion disposed on the distal end of the shaft. The method is comprising: holding the treatment portion in contact with a bone to determine a position where the bone tunnel is to be formed; causing the treatment portion to vibrate in a direction of a longitudinal axis of the shaft while holding the treatment portion in contact with the bone, thereby the bone tunnel is formed; and producing burying small particle of bone upon formation of the bone tunnel in a bone wall at an area of a surface forming the bone tunnel wherein the area extending along a direction of vibrations of the treatment portion.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. An ultrasonic surgical instrument comprising:
an elongated shaft having respective proximal and distal end sides, an ultrasonic transducer being secured to the shaft via the proximal end side, the ultrasonic transducer configured to generate and to transmit ultrasonic vibrations from the proximal end side toward a distal end side along a longitudinal axis of the shaft;
a cutting portion disposed on the distal end side of the shaft, the cutting portion includes an outermost shape-defining portion that defines an outermost shape having a block shape that when pressed at a tip of the block shape against a bone while in a fluid with the ultrasonic vibrations being transmitted thereto, cuts the bone in a direction of a pressing force to form a bone tunnel and to produce small particle of bone at the tip; and
a burying portion disposed on the proximal end side of the outermost shape-defining portion and configured to direct a flow of the fluid, which contains the small particle of bone produced upon formation of the bone tunnel, toward a wall formed in the bone tunnel by the cutting portion along the direction of the pressing force and to bury the small particle of bone in the wall of the bone tunnel.

2. The ultrasonic surgical instrument of claim 1, wherein
the tip of the cutting portion while the ultrasonic vibrations being transmitted, induces the flow of the fluid in a direction along a vibrating direction of the ultrasonic vibrations, and
the burying portion deflects the flow of the fluid in a direction different from the vibrating direction of the ultrasonic vibrations.

3. The ultrasonic surgical instrument of claim 1, wherein
the burying portion is disposed on an outer circumferential surface of the cutting portion at a location on the proximal end side.

4. The ultrasonic surgical instrument of claim 1, wherein
the burying portion is disposed on an outer circumferential surface of the cutting portion at a location on the proximal end side of the outermost shape-defining portion.

5. The ultrasonic surgical instrument of claim 1, wherein
the burying portion has one or more recessed portions extending inward of the cutting portion relative to the outermost shape-defining portion and configured to direct the flow of the fluid, which contains the small particle of bone produced upon formation of the bone tunnel, by the ultrasonic vibrations toward the wall formed by the outermost shape-defining portion.

6. The ultrasonic surgical instrument of claim 5, wherein
the one or more recessed portions have a surface area that intersects the vibrating direction of the ultrasonic vibrations and induces cavitation in the one or more recessed portions.

7. The ultrasonic surgical instrument of claim 6, wherein:
the surface area intersects the vibrating direction at a right angle at the area that induces the cavitation.

8. The ultrasonic surgical instrument of claim 5, wherein
the one or more recessed portions include a channel communicating between the one or more recessed portion and the tip of the cutting portion.

9. The ultrasonic surgical instrument of claim 8, wherein
the channel includes a first opening that opens toward a bottom surface of the bone tunnel, a second opening that opens toward the wall, and a channel surface that intersects the vibrating direction of the ultrasonic vibrations.

10. The ultrasonic surgical instrument of claim 5, wherein
the one or more recessed portions include an outer edge opposing the wall formed in the bone tunnel.

11. The ultrasonic surgical instrument of claim 10, wherein
the outer edge is formed with an outer shape and size so that a portion of the small particle of bone produced upon formation of the bone tunnel is received in the one or more recessed portions.

12. The ultrasonic surgical instrument of claim 5, wherein
the recessed portions are arranged along the direction of the pressing force.

13. The ultrasonic surgical instrument of claim 1, wherein
the cutting portion is configured to form the bone tunnel with an opening having a shape corresponding to a shape of the outermost shape-defining portion.

14. The ultrasonic surgical instrument of claim 1, wherein
the cutting portion includes a tip surface that intersects the vibrating direction of the ultrasonic vibrations and wherein the tip surface is configured to cause cavitation to induce a flow in the fluid.

15. The ultrasonic surgical instrument of claim 14, further comprising:
a filter disposed in the cutting portion and configured to permit the small particle of bone produced upon formation of the bone tunnel to pass through the filter together with the flow of the fluid induced by the cavitation.

16. The ultrasonic surgical instrument of claim 1, wherein
the outermost shape-defining portion with the ultrasonic vibrations being transmitted to the cutting portion forming the wall in the bone tunnel.

17. An ultrasonic surgical assembly comprising:
an ultrasonic surgical instrument having
an elongated shaft including respective proximal and distal end sides, an ultrasonic transducer being secured to the shaft via the proximal end side, the ultrasonic transducer configured to generate and to transmit ultrasonic vibrations from the proximal end side toward a distal end side along a longitudinal axis of the shaft,
a cutting portion disposed on the distal end side of the shaft, the cutting portion includes an outermost shape-defining portion that defines an outermost shape having a block shape that, when pressed at a tip of the block shape against a bone while in a fluid with the ultrasonic vibrations being transmitted thereto, cuts the bone in a direction of a pressing force to form a bone tunnel and to produce small particle of bone at the tip, and
a burying portion disposed on the proximal end side of the outermost shape-defining portion and configured to direct a flow of the fluid, which contains the small particle of bone produced upon formation of the bone tunnel, toward a wall formed in the bone tunnel by the cutting portion along the direction of the pressing force and to bury the small particle of bone in the wall of the bone tunnel; and
an ultrasonic vibration generating section attached on a proximal end side of the ultrasonic surgical instrument.

18. A method of operating an ultrasonic surgical instrument for forming a bone tunnel using an ultrasonic device having a shaft wherein the shaft includes a distal end and a proximal end, and a treatment portion disposed on the distal end of the shaft, the method comprising:
holding the treatment portion in contact with a bone to determine a position where the bone tunnel is to be formed;
causing the treatment portion to vibrate in a direction of a longitudinal axis of the shaft while holding the treatment portion in contact with the bone, thereby the bone tunnel is formed; and
producing burying small particle of bone upon formation of the bone tunnel in a bone wall at an area of a surface forming the bone tunnel wherein the area extending along a direction of vibrations of the treatment portion.
